(19) United States
(12) Patent Application Publication
(10) Pub. No.: US 2023/0221335 A1
(43) Pub. Date: Jul. 13, 2023

(54) A METHOD OF ASSESSING A FEMALE'S RISK OF HAVING PCOS AS WELL AS PRODUCTS AND USES RELATING THERETO

(71) Applicant: Roche Diagnostics Operations, Inc., Indianapolis, IN (US)

(72) Inventors: Sibylle Berner-Gatz, Penzberg (DE); Martin Hund, Rotkreuz (CH); Katharina Buck, Penzberg (DE)

(21) Appl. No.: 17/916,251

(22) PCT Filed: Mar. 30, 2021
(86) PCT No.: PCT/EP2021/058237

§ 371 Date: Sep. 30, 2022

(30) Foreign Application Priority Data
Mar. 31, 2020 (EP) 20167096.5

(51) Int. Cl. G01N 33/68; G16H 50/20; G16H 50/30

(52) U.S. Cl. CPC G01N 33/689; G16H 50/20; G16H 50/30

(57) ABSTRACT

The present invention relates to a method of assessing a female’s risk of having polycystic ovary syndrome (PCOS), a kit for use in assessing a female’s risk of having PCOS, the use of a marker combination in the assessment of a female’s risk of having PCOS, a computer system for use in a method according to the present invention as well as a computer program and a computer-readable storage medium comprising instructions, which when executed by a computer, cause the computer to carry out the method of the present invention.

The present invention relates to a method of assessing a female’s risk of having polycystic ovary syndrome (PCOS), a kit for use in assessing a female’s risk of having PCOS, the use of a marker combination in the assessment of a female’s risk of having PCOS, a computer system for use in a method according to the present invention as well as a computer program and a computer-readable storage medium comprising instructions, which when executed by a computer, cause the computer to carry out the method of the present invention.

Polycystic ovary syndrome (PCOS) is one of the most common endocrine and metabolic disorders affecting 8-13% of reproductive-aged women with up to 70% of affected women remaining undiagnosed. PCOS is a heterogeneous disorder that is defined by a combination of signs and symptoms of androgen excess and ovarian dysfunction. Women with PCOS present with diverse features including psychological (anxiety, depression, body image), reproductive (irregular menstrual cycles, hirsutism, infertility and pregnancy complications) and metabolic features (insulin resistance (IR), metabolic syndrome, prediabetes, type 2 diabetes (DM2) and cardiovascular risk factors) (Escobar-Morreale, H. F. 2018; International evidence-based guideline for the assessment and management of polycystic ovary syndrome 2018).

For a final diagnosis of PCOS, other conditions or diseases should be excluded such as pregnancy, non-classical adrenal hyperplasia (NCAH), androgen secreting tumors, Cushing syndrome, thyroid disorders, or hyperprolactinemia. Diagnostic tests which may be used to exclude other diseases are e.g.
17alpha-Hydroxyprogesterone (17-OHP) to exclude NCAH (Nordenstrom and FalhamMarch 2018)
Prolactin to exclude hyperprolactinemia
Cortisol to exclude patients suffering from Cushing’s syndrome
Thyroid Stimulating Hormone (TSH) to exclude thyroid disorders

PCOS may be caused by a combination of genetic, epigenetic and environmental factors, such as inheritance.

Currently, there is no specific PCOS medication available. Treatment is symptom-oriented and adapted to personal needs. Therapeutic approaches target hyperandrogenism, irregular cycles and associated metabolic disorders. The International evidence-based guideline for the assessment and management of polycystic ovary syndrome 2018 provides information to support clinical decision making and patient management

An option for the diagnosis of PCOS is the so-called Rotterdam Criteria, which is most widely used for definition. PCOS is indicated, if at least 2 of the following criteria apply: (i) irregular cycles and ovulatory dysfunction (oligo-anovulation, OA), (ii) clinical and/or biochemical hyperandrogenism (HA) and (iii) polycystic ovarian morphology (PCOM) (PCOS Consensus Workshop Group, Fertil Steril 2004: 81.19-25). PCOM is usually determined according to the “International Evidence-based Guideline for PCOS 2018” using endovaginal ultrasound transducers with a frequency bandwidth that includes 8 MHz. The threshold for PCOM is considered to be on either ovary: a follicle number per ovary of > 20 and/or an ovarian volume ≥10 ml, ensuring no corpora lutea, cysts or dominant follicles are present. If older ultrasound technology is used, the threshold for PCOM could be an ovarian volume ≥10 ml or a follicle count of >12 on either ovary However, the necessity to consider the results of multiple diagnostic tests and the results of clinical examination needs specific expertise which makes it quite difficult for less specialized physicians (such as general practitioners) to diagnose PCOS in clinical routine. For example, the determination of PCOM by transvaginal ultrasound requires adequate ultrasound equipment and the subjective analysis of ultrasound images by a physician. Furthermore, the result may also depend from the specific ultrasound device used in the assessment of PCOM Consequently, the diagnosis of PCOS based on the Rotterdam Criteria always includes at least one subjective, device- and operator-dependent and error-prone measurement.

Another method for detecting PCOS is measuring the Anti-Mullerian Hormone (AMH) in a subject. AMH is a glycoprotein hormone whose expression is critical to sex differentiation at a specific time during fetal development Further, AMH produced by granulosa cells of growing follicles usually correlates with the number of antral follicles within the ovary. Therefore, serum levels of AMH may be a surrogate biomarker for the antral follicle count/number (AFC) determined by transvaginal ultrasound. Some studies have suggested serum AMH as biochemical marker for PCOM . In small studies, AMH threshold values for PCOM in women with PCOS were proposed (Nicholas at al. 2014; Pigny et al. 2016). However, according to the “International evidence-based guideline for the assessment and management of polycysticovary syndrome 2018° serum AMH levels should not be used as an alternative for the detection of PCOM or to diagnose PCOS.

A further method for detecting PCOS is a 3-item PCOS criteria system (indran et al, 2018). In this system, it was proposed that diagnosis of PCOS is made, if two out of three items are present: (i) oligomenorrhea (defined as mean menstrual cycle length > 35 days); (ii) AMH above threshold, and (iii) hyperandrogenism defined as either testosterone above threshold and/or the presence of hirsutism (mFG score ≥ 5). Alternatively, AMH was suggested in combination with hyperandrogenism and oligomenorrhea (Sahmay et al., 2014) or in combination with SHBG (Calzada et al., 2019).

The Russian application RU2629720 suggests a method for predicting the level of risk of development of polycystic ovary syndrome (PCOS) in adolescent girls considering a huge list of indicators from the fields of anamnestic signs, clinical signs, laboratory signs and echography signs. In total, these fields comprise 33 different values that are all determined from an adolescent girl patient and family members and then, depending on their importance, weighted with a factor of 1, 2 or 3 points. The weighted values are added up and the resulting total of points indicates a low, medium or high level of risk of development of polycystic ovary syndrome.

So far, there is no universal test available indicating PCOS. Additional hormones are often tested to evaluate a woman with suspicion of PCOS such as e.g. luteinizing hormone (LH) and follicle-stimulating hormone (FSH). However, the diagnostic utility of the LH:FSH ratio for the diagnosis of PCOS seems low as only a small percentage of women with PCOS have significantly elevated LH:FSH ratios (Cho et al. 2005). Actually, there is a wide range of LH:FSH ratios found in women diagnosed with PCOS (Malini and George 2018).

Further, there is a need for a “decision support system” helping the physician to identify women with high risk of having PCOS. Especially, there is a need of a less error-prone and objective method for assessing a female’s risk of having PCOS. The method is preferably simple to be carried out and does not involve subjective assessments.

Accordingly, the object of the present invention is to provide a more accurate and/or objective method of assessing a female’s risk of having PCOS which is less error-prone than the methods of the state of the art Preferably, the method is a computer-implemented method.

In a first aspect, the invention relates to a method of assessing a female’s risk of having polycystic ovary syndrome (PCOS), the method comprising
a) providing a data set including
   an OA-vaiue reflecting the length of the female’s menstrual cycle and/or the number of the female’s menstrual cycles per year, wherein an increased OA-value relative to the OA-value of a healthy reference population indicates an abnormal menstrual cycle length and/or number,
   a HA~value reflecting the female’s androgen status, wherein an increased HA--value relative to the HA-vaiue of a healthy reference population indicates an increased androgen level in the female, and
   an AMH-vaiue corresponding to the amount or concentration of anti-Mullerian hormone (AMH) in a sample obtained from the female;
b) processing the data set provided in step at with a processing unit, wherein the processing comprises combining values of the data set provided in step a) into one combined value;
c) comparing the combined value obtained in step b) to the corresponding combined value as established in a reference population, wherein an increased combined value of the female relative to the combined value of a healthy reference population is indicative of an increased risk of PCOS: and
d) indicating the female’s risk of having PCOS via an indication unit

As shown by the Examples, the method of the present invention can be used for the assessment of a females risk of having PCOS. In the Examples, a reference population including healthy women (controls) as well as those diagnosed with PCOS (cases) was used to establish a regression model. For this,the women’s (cases and controls) data relating to the menstrual cycle, the ratio of the concentration of total testosterone (TT), the concentration of sex hormone-binding globulin (SHBG) and the concentration of AMH were collected in a data set and translated into the OA-, HA- and AMH-values, Optionally, the data set also included the female’s body weight and height, and the female’s age, which were translated into a WEIGHT-value and an AGE-value, respectively. These values were combined into a single value indicating the female’s risk of having PCOS step A weighted logistic regression model was established wife case-control status as endpoint within a Monte-Carlo cross-validation (MCCV)Results indicate that OA has the largest influence on the PCOS risk followed by AMH and Free Androgen Index (FAI FAI=totat testosterone X 100 / SHBG). Small standard deviations indicate quite stable regression coefficients throughout the MCCV runs. The inventors also found that the selection of suitable variables is important, as other variables (e.g. antral follicle count, LH and FSH) were found less suitable in the present method. It is evident that PCOS is a syndrome which is characterized by a set of symptoms, wherein each of the symptoms may or may not be present in a single female and when present may be present in a different extent. However, when looking at the combined value according to the present invention, females diagnosed with PCOS have been proven of having an increased combined value when compared to females without PCOS.

The examples further provide a scoring system, allowing the classification of a subject into a low, moderate or high risk for PCOS. Beside the numerical score, this further allows the visualization of a female’s risk of having PCOS (e.g. red, yellow, green).

Each of the values included into the data set may be determined at a numerical level (cycle length/number, hormone concentrations or amounts, weight and age), Contrary to the present methods, in which the ovarian morphology is usually characterized by a physician, the method of the invention for assessing the risk status of a parent for PCOS does not comprise a mandatory value (i.e.OA-value. HA-value and AMH-value optionally in combination with AGE-value and WEIGHT-value) that needs to be subjectively determined (i.e., demanding personal judgement), thereby allowing the reduction of susceptibility to errors. . Further, the method of the invention allows standardized large-scale examinations of women. Moreover, the data set processed in step b) does not include data from a person other than the assessed female (such as family members, e.g. the mother) or data rejecting the past rather than the presence (i.e. time before sexual maturity), such as the female’s birth weight. Such parameters are prone to errors, Including such parameters may result in false negative characterization. In this respect, it is referred to in RU2629720. A female fulfilling all diagnostic criteria for PCOS: oligo-anovulation (OA) and hyparandrogenism (HA) and polycystic ovarian morphology (PCOM) would be incorrectly classified as low risk with the method of RU2629720, if there was no (family) history of PCOS and if she did not show further clinical symptoms (hirsutism or acne).

The female’s risk of having PCOS can be assessed even more accurately by applying the method of the present invention, whereby the data set provided in step a) does not only include an OA-value, a HA-value and an AMH-value, but also a WEIGHT-value reflecting the female’s body weight; and/or an AGE-value reflecting the female’s age Accordingly, in a preferred embodiment of the present invention data set provided in step a) includes an OA-value, a HA-value, an AMH-value and an AGE-value. More preferably, the data set provided in step a) includes an OA-value, a HA-value, an AMH-value, an AGE-value and a WEIGHT-value.

If a female should have been identified as having an increased risk for PCOS, she may be suggested for a differential diagnosis or monitoring with respect to PCOS, including any of those mentioned above, e.g. the analysis of the ovaries e.g. by ultrasound in order to detect PCOM and in order to exclude or confirm PCOS. Moreover, other diseases such as non-classical adrenal hyperplasia (NCAH), androgen secreting tumors, Cushing syndrome, thyroid disorders, or hyperprolactinemia should be excluded. Additionally, clinical symptoms of PCOS such as infertility, and PCOS-influenced disorders/diseases such as insulin resistance and/or diabetes may be assessed and treated in an appropriate way based on the diagnosis of PCOS.

As detailed above, the method according to the first aspect of the invention may be used for assessing a female’s risk of having PCOS.

In this matter, the term “assessing a female’s risk of having PCOS” describes the analysis of the probability that the female examined by the method of the first aspect may have or develop (preferably has) PCOS. The result of the analysis may be qualitative or quantitative. This means that the result may be either that the female has or has not a risk of having PCOS (qualitative) or the risk may be further defined as e.g. high, moderate or low (quantitative). In the latter case, the female’s risk may be defined by a numerical value such as a percentage value specifying the risk in the range of 0% to 100% or a risk score having a value within a given range, such as between 0 to 2 (0=low; 1=moderate; 2=high), or 0 to 9 (0-2=low; 3-5=moderate; 6-9=high), or the like

The term “female” describes the sex of an organism that provides oocytes. The female may be of any species, for example, the female may be preferably a female mammal, for example, a human, a horse, a cat or a dog. More preferably, the female is a human. In a preferred embodiment, the female is a human. If the female is a mammal, such as a human, it may be characterized by two X-chromosomes. The female may be sexually mature. Preferably, the female is in the reproductive age, i.e. after the beginning of fertility and before menopause. If the female is a human, it may be from 10 to 60 years old, preferably from 13 to 55 years old, more preferably from 15 to 50 years old and most preferably from 18 to 45 years old

The female examined by the method according to the first aspect of the invention may not have PCOS at all or may have PCOS or develop any form or degree of PCOS in the future, such as women with PCOS showing mild or severe symptoms. Further, the female examined by the method according to the first aspect of the invention may have any phenotype of PCOS or any combination of symptoms associated with PCOS. Symptoms or phenotypes are well-known to the person skilled in the art.

Step a) according to the method of the first aspect of the invention, comprises providing a data set including
an OA-value reflecting the length of the female s menstrual cycle and/or the number of the female’s menstrual cycles per year, wherein an increased OA-value relative to the OA-value of a healthy reference population indicates an abnormal menstrual cycle length and/or number,
a HA-value reflecting the female’s androgen status, wherein an increased HA-value relative to the HA-value of a healthy reference population indicates an increased androgen level in the female, and
an AMH-value corresponding to the amount or concentration of anti-Müllerian hormone (AMH) in a sample obtained from the female.

The term “OA-value” describes a value reflecting the length of the female’s menstrual cycle and/or the number of the female’s menstrual cycles per year and reflects oligomenorrhea and/or anovulation (OA), An increased OA-value relative to the OA-value of a reference population indicates an abnormal menstrual cycle length and/or number, which is indicative of an increased risk of having PCOS. Usually, the OA-value directly or indirectly correlates with the length of the female’s menstrual cycle and/or the number of the female’s menstrual cycles per year as well as with oligomenorrhea and/or anovulation. A female having a normal length of the menstrual cycle and a normal number of menstrual cycles per year is referred to as symptom-free with respect to OA. Oligomenorrhea and/or anovulation is one symptom of PCOS.

The term “female menstrual cycle” describes the regular natural change that usually occurs in the female reproductive system due to the rise and fall of hormones. These hormonal fluctuations in general result in the growth and thickening of the endometrium as well as the growth of an egg. The egg may be released from an ovary around day fourteen in the cycle. If pregnancy does not occur, the endometrium is released in what is known as menstruation.

The term “menstruation” describes the discharge of blood, secretion and mucosal tissue (known as menses) from the inner lining of the uterus through the vagina. The blood may be liquid or coagulated.

The length of the female’s menstrual cycle may be determined as follows:
the first day of the menstruation in general is considered to be the first day of a female menstrual cycle and
the female menstrual cycle is usually considered to end with the last day before the beginning of the next menstruation.

In general, the human female menstrual cycle lasts from 21 to 35 days. Usually, a female has 11 to 13 menstrual cycles per year (for adult women > 3 years after menarche and not using any form of hormonal contraceptive). The term “oligomenorrhea” describes the condition that the cycle length of a woman is repeatedly >35 days. Repeatedly means that this occurs often or always and does not result from other circumstances, such as a disease other than PCOS such as e.g. anorexia. Preferably, in oligomenorrhea the female menstrual cycle lasts >40 days, more preferably >50 days and mostly preferred >60 days. In oligomenorrhea, the female menstrual cycle may even last up to 90 days. It is well-known in the art that the cycle length may vary from cycle to cycle in individual females. Thus, oligomenorrhea may also describe the condition that a female has <8, preferably <6 and more preferred <4 menstrual cycles per year.

The term “anovulation” usually describes the condition when the ovaries do not release any oocyte during a female menstrual cycle at all. The female whose risk of having PCOS is assessed may be determined to suffer from anovulation, if no oocyte is released for the duration of at least one female menstrual cycle, preferably at least three female menstrual cycles, more preferably at least six female menstrual cycles and mostly preferred at least nine female menstrual cycles in one year. Further, the female whose risk of having PCOS is assessed may be determined to suffer from anovulation, if no oocyte is released for the duration of at least 6 months, preferably at least 9 months, more preferably at least 1 year.

Information about oligomenorrhea or anovulation may simply be gathered by asking or monitoring the female. By e.g. keeping a calendar, where details about the beginning and end of menstruation are entered, this information usually is very accurate. Oligomenorrhea and/or anovulation are translated into an increased OA-value relative to the OA-value of females with normal menstrual cycle. Therefore, an increased OA-value relative to the OA-value of a reference population is considered to indicate an abnormal menstrual cycle length and/or number, which is indicative of an increased risk of having PCOS.

As detailed above, values defining normal and abnormal menstrual cycle lengths and numbers are well-known in the art. Values defining normal menstrual cycle lengths and numbers may be obtained from a healthy reference population or from standard publications. The term “healthy reference population” describes a population of apparently healthy females not suffering from PCOS or any disease affecting the female menstrual cycle or the amount or concentration of sexual hormones.

For example, the menstrual cycle lengths and / or numbers of females 1) belonging to the healthy reference population or 2) suffering from PCOS are analyzed. This may lead to a widespread data set of values associated to the health condition of a female, e.g. females being apparently healthy or women with PCOS having various forms or severities of PCOS symptoms allowing establishing cut-off values to differentiate the groups.

Alternatively, no thresholds may be applied, but a continuous correlation may be used instead, i.e. a high deviation of a menstrual cycle from the healthy reference population may then correspond to a high OA-value, while a low deviation of a menstrual cycle from the reference population may correspond to a low OA-value.

Further, the value used for determining the OA-value, such as the female’s cycle length, may be subject of any mathematical operation before the OA-value is determined. Suitable mathematical operations are well-known to the person skilled in the art and comprise, for example, addition, subtraction, multiplication, division or logarithmising.

Moreover, the OA-vaiue (X_{OA}) may for example be determined by grouping of values (e.g. by forming percentiles) and determining cut-off values.

For example, if the menstrual cycle lengths and / or numbers are grouped into two groups, the group of the healthy reference population composed of females without PCOS may be allocated a minimum number for X_{OA}, such as X_{OA} = 0, while the group of females having any form of oligomenorrhea or anovulation may be allocated any other number for X_{OA}, such as a 1 or 2.

For human females, the OA-value (X_{OA}) may, for example, be determined as follows:
X_{OA} = 2, if the female suffers from oligomenorrhea and anovulation (e.g. has no menstrual cycle; OR if the average menstrual cycle length >35 days, preferably >40 days, more preferably >50 days and mostly preferred >60 days); OR if the female has <8 cycles per year, more preferably <6 cycles per year and mostly preferred <4 cycles per year,
X_{OA} = 0, if the female is symptom-free (i.e. does not suffer from oligomenorrhea and anovulation)

In the method of the invention, the female’s data on her menstrual cycle will be translated into an OA-value considering the threshold or cut-off value chosen to differentiate between the groups (e.g., with and without symptoms).

Moreover, if the menstrual cycle lengths and / or numbers are categorized in more than two groups (e.g. without symptom, with symptom oligomenorrhea and with symptom anovulation), symptom-free females may be allocated a minimum number, (such as X_{OA} = 0), while a female having oligomenorrhea or even anovulation may be allocated a higher numbers (such as X_{OA} = 1 or 2, respectively).

Most preferably, the OA-value is a categorical variable for oligo-/anovulation (OA) (yes, no). Oligo-/anovulation is assumed, if the menstrual cycle length is reported as repeatedly > 35 days (see Example 1).

The term “HA-value” describes a value reflecting the female’s androgen status, wherein an increased HA-value relative to the HA-value of a healthy reference population indicates an increased androgen level in the female which is regarded as PCOS symptom. Usually, the HA-value directly correlates with the female’s androgen status. A female having a normal female androgen status is referred to as symptom-free with respect to HA. An increased level of an androgen in a female is one possible symptom of PCOS.

Increased levels of androgens in females are referred to as “hyperandrogenemia (HA)”. Phenotypical symptoms may include e.g. acne, seborrhea (inflamed skin), hair loss on the scalp, increased body or facial hair, and infrequent or absent menstruation. Hyperandrogenemia may be a diagnostic feature of PCOS, comprising potentially both conditions, clinical (hirsutism, alopecia and acne) and biochemical hyperandrogenemia. Hyperandrogenemia may, among others, be caused by PCOS,

In the present invention, the HA-vaiue directly correlates with the female’s androgen status,

The term “androgen” describes any natural or synthetic steroid hormone that regulates the development and maintenance of male characteristics in vertebrates by binding to androgen receptors. Androgens are usually synthesized in the testes, the ovaries, and the adrenal glands. Androgens generally increase in both boys and girls during puberty. Also, androgens are the precursors to estrogens in both men and women. Examples for androgens involved in the female menstrual cycle are dehydroepiandrosterone (DHEA), dehydroepiandrosterone sulfate (DHEA-S), androstenedione, testosterone and dihydrotestosterone (DHT).

In general, testosterone is the main androgen in male. Females usually produce much lower amounts of testosterone than males, which affects growth and maintenance of female reproductive tissue and bone mass. Exemplified for the Elecsys Testosterone II assay the normal concentration of total testosterone in women aged 20-49 years in blood is considered to be between 0.08-0.48 ng/mL (corresponding to 0.29-1.67 nmol/L in blood). However, the concentration of testosterone produced in the body may vary each day and throughout the day, e.g. usually, testosterone concentrations are highest in the morning. Further, testosterone concentrations may depend on age or health history. It is also well-known in the art that the reference ranges depend on assays and methodologies used; reference ranges for apparently healthy individuals are determined by the provider and given in the assay description/method sheet/package insert.

In the method according to the first aspect of the present invention, the female’s androgen status is determined by measuring a biochemical parameter, namely an androgen, in the female’s sample. Preferably, the amount or concentration of free testosterone (FT) in a sample obtained from the female, or the ratio of the amount or concentration of total testosterone (TT) and the amount or concentration of sex hormone-binding globulin (SHBG) in a sample obtained from the female (TT/SHBG), optionally multiplied by a constant a (a * TT/SHBG), especially multiplied by 100 (100 * TT/SHBG) is determined.

The term “total testosterone” describes all three types of testosterone in the blood: testosterone attached to another molecule (such as a protein like albumin or sex hormone binding globulin (SHBG) as well as testosterone that is not attached to any other molecule, particularly protein, (free testosterone)) Usually, testosterone in human circulates in the bloodstream, loosely bound mostly to serum albumin and to sex hormone binding globulin (SHBG), Only a very small fraction of testosterone is unbound, or “free,” and thus biologically active and able to enter a cell and activate its receptor. In addition, also testosterone that is weakly bound to albumin is bioavailable and can be readily taken up by the body’s tissues.

In general, a total testosterone test does not distinguish between bound and unbound testosterone, but determines the overall quantity of testosterone. Methods for measuring total testosterone are well-known to the person skilled in the art. For example, a needle may be used to draw blood from a vein in arm or hand. Suitable methods for detecting total testosterone are, for example, an immunoassay and / or mass spectrometry, such as liquid chromatography-mass spectrometry (LCMS) / mass spectrometry, enzyme-linked immunosorbent assay (ELISA), electrochemiluminescence-immunoassay (ECLIA), and extraction / chromatography immunoassays, preferably electrochemiluminescence-immunoassay (ECLIA), such as Elecsys® from Roche. Due to the varying testosterone concentrations throughout the day, this test is usually performed in the morning and may further be repeated several times to gain more accurate values, which may then be statistically evaluated, e.g. by forming a mean or median value or other statistical techniques well-known to the person skilled in the art

Moreover, in the method according to the first aspect of the present invention, the female’s androgen status may be determined by e.g., determining the Free Androgen Index (FAI) The free androgen index usually is intended to give a guide to the free testosterone concentration (Vermeulen et al. 1999).

Preferably, the FAI is determined for determining the HA-value.

The term “Free Androgen index (FAI)” describes a ratio used to determine abnormal androgen status in humans. The ratio, calculated on a molar/molar basis, is the total testosterone concentration divided by the sex hormone binding globulin (SHBG) concentration, and then multiplying by a constant, usually 100:

$$\text{FAI \%: Total Testosterone} \text{/} \text{SHBG} \times \text{100} \text{.}$$

Methods for measuring total testosterone are mentioned above,

SHBG is a glycoprotein that binds to androgens and estrogens and thereby inhibits the function of these hormones. Thus, bioavailability of sex hormones is influenced by the concentration of SHBG. Methods for measuring SHBG are well-known to the person skilled in the art. For example, a needle may be used to draw blood from a vein in arm or hand. Methods for the detection of SHBG are e.g., an immunoassay and / or mass spectrometry, such as liquid chromatography-mass spectrometry (LCMS) / mass spectrometry, enzyme-linked immunosorbent assay (ELISA), electrochemiluminescence-immunoassay (ECLIA), and extraction / chromatography immunoassays, preferably electrochemiluminescence-immunoassay (ECLIA), such as Elecsys® from Roche.

Exemplified for the Elecsys SHBG assay, a premenopausal adult female (human) has an SHBG concentration of 32-128 nmol/L while a pubertal female (human) has an SHBG concentration of 36-125 nmol/L in blood. At the age or 50 or older, the SHGB concentration of a female (human) is about 27-128 nmol/L.

Increased levels of androgens (such as an increased amount or concentration of free testosterone (FT) in a sample obtained from the female, or the ratio of the amount or concentration of total testosterone (TT) and the amount or concentration of sex hormone-binding globulin (SHBG) in a sample obtained from the female) are translated into an increased HA-value relative to the HA-value of females with normal androgen status/levels. Therefore, an increased HA-value relative to the HA-value of a reference population is considered to indicate an increased androgen level in the female, which is indicative of an increased risk of having PCOS.

As detailed above, values defining normal and abnormal androgen status are well-known in the art. Values defining normal androgen status may be obtained from standard publications or from a reference population (see also above with respect to the OA-value).

For human females, the HA-value (X_{HA}) may for example be determined based on the FAI or the FT. It is possible to apply a continuous correlation, i.e. a high FAI or FT may then correspond to a high HA-value, while a low FAI or FT may correspond to a low HA-value.

Further, the value used for determining the HA-value, such as the FAI or the FT, may be subject of any mathematical operation before the HA-value is determined Suitable mathematical operations are well-known to the person skilled in the art and comprise, for example, addition, subtraction, multiplication, division or logarithm ising.

Moreover, the HA-value (X_{HA}) may for example be determined by grouping of values (e.g. by forming percentiles) and determining cut-off values. Cut-off values, grouping, translation of values etc. may be defined as described above with respect to the OA-value.

For human females, the HA-value (X_{HA}) may for example be determined as follows:
X_{HA} = 2, if female shows an increased androgen level (e.g. the FAI > threshold (whereby the threshold may be e.g., 5.5%, preferably 6%, more preferably 6.5%, mostly preferred 7%) AND/OR total testosterone > threshold (whereby the threshold may be e.g., 48 ng/dl, preferably 52 ng/dl, more preferably 56 ng/dl, mostly preferred 60 ng/dl));
X_{HA} = 0, if the female is symptom-free (i.e. does not show an increased androgen level)

Most preferably, the HA-value is a numeric variable for HA. Hyperandrogenism (HA) is derived as the free androgen index (FAI) calculated on a molar/molar basis from levels of serum testosterone (nmol/l) and serum sex hormone-binding globulin (SHBG) (nmol/l), wherein FAI = testosterone / SHBG * 100 (see Example 1).

The term “AMH-value” describes the amount or concentration of anti-Müllerian hormone (AMH) in a sample obtained from the female. An increased AMH-value relative to the AHM-value of a reference population indicates an increased amount or concentration of anti-Mullerian hormone (AMH) in the subject, which is regarded as PCOS symptom. Usually the AMH-value directly correlates with the amount or concentration of anti-Muilerian hormone (AMH) in a sample obtained from the female. A female having a normal amount or concentration of AHM is referred to as PCOS symptom-free with respect to AMH. An increased amount or concentration of AMH in a female is one possible symptom of PCOS.

The term “amount” describes a standards-defined quantity of a substance that measures the size of an ensemble of elementary entities, such as atoms, molecules, electrons, and other particles. It is sometimes referred to as chemical amount. The International System of Units (SI) defines the amount of substance to be proportional to the number of elementary entities present. The Sl unit for amount of substance is the mole. It has the unit symbol mol.

The “concentration” of a substance is the amount of a constituent divided by the total volume of a mixture. Several types of mathematical description can be distinguished: mass concentration, molar concentration, number concentration, and volume concentration. The term concentration can be applied to any kind of chemical mixture, but most frequently it refers to solutes and solvents in solutions. The molar (amount) concentration has variants such as normal concentration and osmotic concentration.

The term “sample” describes any kind of fluid or tissue obtained from a female. The sample may be any sample suitable for measuring the marker(s) according to the present invention, such as AMH or androgen, and may refer to a biological sample obtained for the purpose of evaluation in vitro. The sample may comprise material which can be specifically related to the individual and from which specific information about the individual can be determined, calculated or inferred. Exemplary samples include blood, serum, plasma or urine.

Preferably, the sample is a blood sample, more preferably selected from the group consisting of serum, plasma, and whole blood.

The sample may be obtained by any method for obtaining a sample from the female body known to the person skilled in the art. For example, a needle may be used to draw blood from a vein in arm or hand from the female.

Methods for detecting the amount or concentration of AMH in a sample from a female are well-known to the person skilled in the art. For example, AMH amount or concentration in serum may be detected using an immunoassay and / or mass spectrometry, such as liquid chromatography-mass spectrometry (LCMS) / mass spectrometry, enzyme-linked immunosorbent assay (ELISA), electrochemiluminescence-immunoassay (ECLIA) and extraction / chromatography immunoassays, preferably electrochemiluminescence-immunoassay (ECLIA), such as Elecsys® from Roche.

Usually, women (human) have an age-dependent concentration of AMH. Examples are shown in Table 1.

TABLE 1

Age-dependent AMH concentrations in the blood of men and women.

| | N | 2.5^{th} perc. ng/ml (95 % Cl^{b}) | 5^{th} perc ng/ml (95 % Cl) | Median ng/ml (95 % Cl) | 95^{th} perc. ng/ml (95 % Cl) | 97.5th perc. ng/ml (95 % Cl) |
|---|---|---|---|---|---|---|
| Healthy men | | | | | | |
| | 148 | 0.77 (0.17-1.58 ) | 1.43 (0.256-1.97) | 4.79 (4.35-5.35) | 11.6 (10.3-17.0) | 14.5 (10.9-17.6) |

Healthy women (years)

| | | | | | | |
|---|---|---|---|---|---|---|
| 20-24 | 150 | 1.22 (0.478-1.67) | 1.52 (0.758-1.81) | 4.00 (3.60-4.44) | 9.95 (7.87-13.6) | 11.7 (9.11-15.7) |
| 25-29 | 150 | 0.890 (0.493-1.21) | 1.20 (0.797-1.75) | 3.31 (3.00-3.89) | 9.05 (7.59-10.3) | 9.85 (8.91-11.3) |
| 30-34 | 138 | 0.576 (0.256-0.958) | 0.711 (0.256-1.12) | 2.81 (2.35-3.47) | 7.59 (6.84-10.3) | 8.13 (7.27-9.72) |
| 35-39 | 138 | 0.147 (0.053-0.474) | 0.405 (0.053-0.496) | 2.00 (1.73-2.36) | 6.96 (5.31-9.37) | 7.49 (6.40-10.9) |
| 40-44 | 142 | 0.027 (0.010-0.063) | 0.059 (0.017-0.119) | 0.882 (0.726-1.13) | 4.44 (2.94-5.56) | 5.47 (3.92-6.76) |
| 45-50 | 169 | 0.010 (0.010-0.010) | 0.010 (0.010-0.010) | 0.194 (0.144-0.269) | 1.79 (1.43-2.99) | 2.71 (1.79-4.16) |

The corresponding amounts of AMH may be calculated by multiplication of the value of the concentration with the volume.

Increased amounts or concentrations of AMH in a sample obtained from the female may be translated into an increased AMH-value relative to the AMH-value of females with normal AMH amounts or concentrations. Therefore, an increased AMH-value relative to the AMH--value of a reference population may be considered to indicate an increased AMH amount or concentration in the female, which is indicative of an increased risk of having PCOS,

As detailed above, values defining normal and abnormal AMH are well-known in the art Values defining normal AMH status may be obtained from standard publications or from a reference population or (see also above with respect to the OA-value).

For human females, the AMH-value (X_{AMH}) may for example be determined based on the amount or concentration of AMH. It is possible to apply a continuous correlation, i.e. a high AMH amount or concentration may then correspond to a high AMH-value, while a low AMH amount or concentration may correspond to a low AMH-value.

Further, the value used for determining the AMH-value, such as the amount or concentration of AMH, may be subject of any mathematical operation before the AMH-value is determined. Suitable mathematical operations are well-known to the person skilled in the art and comprise, for example, addition, subtraction, multiplication, division or logarithmising.

Moreover, the AMH-value (X_{AMH}) may for example be determined by grouping of values (e.g. by forming percentiles) and determining or cut-off values. Cut-off values, grouping, translation of values etc. may be defined as described above with respect to the OA-value.

For human females, the AMH-value (X_{AMH}) may for example be determined as follows:
X_{AMH} = 2, if the female shows an increased AMH level (e.g. if serum AMH > 3.5 ng/ml, preferably > 4 ng/ml, more preferably > 4,5 ng/ml, mostly preferred > 5 ng/ml);
X_{AMH} = 0, if the female is symptom-free (i.e. does not show an increased AHM level)

Most preferably, the AMH-value is a numeric variable for AHM expressed as level of serum AMH (nmol/l) (see Example 1).

The data set of step a) may further comprise other values concerning e.g. further conditions of the female’s body or substances (such as hormones) present in the female body.

Suitable values of the dataset of step a) may also concern further substances present in the female body e.g., estrogens, androgens (such as testosterone (e.g. FT and / or bioavailable testosterone) dehydroepiandrosterone (DHEA), dehydroepiandrosterone sulfate (DHEA-S), androstenedione, and / or dihydrotestosterone (DHT)).

Suitable values of the dataset of step a) concerning further conditions of the female’s body may concern e.g., the female’s body weight, the female’s age and / or phenotypical characteristics.

Further features that may be considered are inheritance, the female’s lifestyle habits, such as smoking or physical exercises, or environmental conditions.

Step b) according to the method of the first aspect of the invention, comprises processing the data set provided in step a) with a processing unit, wherein the processing comprises combining values of the data set provided in step a) into one combined value.

The term “processing unit” describes a component capable of performing a method encoded by an executable code, such as an electronic circuit which performs operations on some external data source, usually memory or some other data stream. For example, the processing unit may be computer or a mobile device, such as a smartphone.

The term “processing” describes the collection and change of items of data or information in any manner to produce meaningful information. Moreover, an app (application) or computer program may be installed on the processing unit. Further, this app may support the processing and further provide a graphical user interface or text-based user interface.

In step b) of the method of the first aspect of the present invention, processing comprises combining the values of the data set provided in step a) (OA-, HA- and AHM-value and optionally WEIGHT- and/or AGE-value) into one combined value. Combining the values of the data set provided in step a) into one combined value may be performed using any mathematical operation known to the person skilled in the art. Moreover, combining the values of the data set provided in step a) into one combined value may also comprise the application of any statistical analysis known to the person skilled in the art. Preferably, the OA-, HA- and AHM-value are combined by addition and the combined value is the sum of these values.

Also preferably, combining the values of the data set provided in step a) into one combined value may comprise the application of a weighting factor for increasing or decreasing the influence of single values on the combined value. This means that one of the values may be given a higher weighting than the other values. The weighting factors may be obtained by analyzing the reference population of females without PCOS (healthy reference population) and / or females having PCOS mathematically. Preferably, the weighting factors may or have been obtained by analyzing a population of females without PCOS and females having PCOS mathematically. More preferably, the weighting factors have been obtained by analyzing a population of females without PCOS and females having PCOS with a weighted regression model, especially, a weighted logistic, regression model. Suitable methods are shown in the Examples.

For example, the values (OA, HA, AHM and optionally AGE and/or WEIGHT) of females 1) belonging to the healthy reference population or 2) suffering from PCOS are analyzed/collected, Weighting factors for the variables may be determined by established mathematical procedures. This may lead to a widespread data set of PCOS risk probabilities (see FIG. 3) associated to the health condition of a female, e.g. females being apparently healthy or having various forms or severities of PCOS allowing establishing cut-off values to differentiate the groups (see FIG. 3), The group of females suffering from PCOS may be further subdivided according to the severity of symptoms in women with PCOS, leading to more than two groups, such as no PCOS, PCOS with mild symptoms and PCOS with severe symptoms, and allowing further differentiation. An exemplary procedure is described in the Examples.

For example, at least one, two or three of the values provided in step a) are weighted by applying a weighting factor. Preferably, values provided in step a) are weighted by applying a weighting factor.

Preferably, in step b) of the first aspect of the invention the combined value is a weighted combined value obtained by weighted calculation of the values provided in step a).

For example, the OA-value, the HA-value and the AMH-value may further be processed by weighting factors W_{OA}, W_{HA} and W_{AMH}, respectively. The weighting factors W_{OA}, W_{HA} and W_{AMH} may be determined by comparing females of a reference population without PCOS to females of a population having PCOS. W_{OA} may reflect the frequency or significance of oligomenorrhea and / or anovulation in PCOS patients. A high frequency or significance of oligomenorrhea and / or anovulation may be correlated to a high W_{OA}, while a low frequency or significance of oligomenorrhea and / or anovulation may be correlated to a low W_{OA} W_{HA} may reflect the frequency or significance of an increased androgen levels in PCOS patients. A high frequency or significance of an increased androgen level may be correlated to a high W_{HA}, while a low frequency or significance of an increased androgen level may be correlated to a low W_{HA}, A high significance of the amount or concentration of AMH may be correlated to a high W_{HA}, while a low significance of the amount or concentration of AMH may be correlated to a low W_{AMH}.

If a weighting factor is applied to each value (OA-value, HA-value and AHM-value), the data set provided in step a) may, for example, be combined using the following algorithm (PCOS Scoring algorithm):

$$\text{Score} \text{=} {\text{X}}_{\text{OA}} * {\text{W}}_{\text{OA}} + {\text{X}}_{\text{HA}} * {\text{W}}_{\text{HA}} + {\text{X}}_{\text{AMH}} * {\text{W}}_{\text{AMH}}$$

whereby X_{OA}, X_{HA} and X_{AMH} may be defined as described above and W_{OA}, W_{HA} and W_{AMH} are weighting factors for weighting X_{OA}, X_{HA} and X_{AMH}.

As shown in the Examples, the weighting factors may be defined considering that: W_{OA} > W_{AMH} and W_{OA} > W_{HA}.

To determine the Score, any interactions between X_{OA}, X_{HA}, X_{AMH}, W_{OA}, W_{HA} and / or W_{AMH} are possible. For example, this also comprises the combination of weighting factors using any interactions, such as

$$\text{Score} \text{=} {\text{X}}_{\text{OA}} * {\text{W}}_{\text{OA}} + {\text{X}}_{\text{HA}} * {\text{W}}_{\text{HA}} + {\text{X}}_{\text{AMH}} * {\text{W}}_{\text{AMH}} + {\text{W}}_{\text{OA HA}} * {\text{X}}_{\text{HA}} * {\text{X}}_{\text{OA}}$$

Moreover, these interactions comprise any mathematical operation known to the person skilled in the art, such as addition, subtraction, multiplication, division or logarithmising.

Step c) according to the method of the first aspect of the invention, comprises comparing the combined value obtained in step b) to the corresponding combined value as established in a reference population, wherein an increased combined value of the female relative to the combined value of the reference population is indicative of an increased risk of PCOS.

The term “reference population” describes a population which may comprise apparently healthy females not suffering from PCOS, in particular any disease affecting the female menstrual cycle or the amount or concentration of sexual hormones as well as females suffering from PCOS or any disease affecting the female menstrual cycle or the amount or concentration of sexual hormones. Preferably, the reference population may be chosen to comprise at least 20, 30, 50, 100, 200, 500 or 1000 individuals. It is within the skills of the practitioner to choose appropriate individuals for the reference population.

Preferably, the combined value obtained in step b) and the combined value of the reference population have been obtained using the same mathematical procedure, e.g. using the same algorithm.

For example, the combined value of a reference population may be calculated as described above for the female assessed for the risk of having PCOS. Therefore, the combined value of all females belonging to this healthy reference population may be assessed and combined as a combined value of a healthy reference population by statistical analysis, such as by determining the mean or median value of the combined values of all females belonging to this healthy reference population. Further suitable methods for statistical analysis are well-known to the person skilled in the art.

The combined value obtained in step b) may be considered to be higher (increased) than the combined value as established in a healthy reference population, if it is significantly higher than the combined value as established in a reference population. Statistical procedures to assess whether two values are significantly different from each other are well-known to the person skilled in the art.

For example, the combined value obtained in step b) may be considered to be higher (increased) than the combined value as established in a healthy reference population, if it is at least by factor 1.1, preferably by factor 1.5, more preferably by factor 2.0 and mostly preferred by factor 2.5 higher than the combined value as established in a healthy reference population.

Moreover, the combined value obtained in step b) may be considered to be higher (increased) than the combined value as established in a healthy reference population, if it is by the absolute value of at least 0.5, preferably at least 1.0, more preferably at least 2.0 and mostly preferred at least 2 5 higher than the combined value as established in a healthy reference population.

Furthermore, the combined value obtained in step b) may be considered to be higher (increased) than the combined value as established in a healthy reference population, if it is increased by at least 10%, in particular by at least 25%, in particular by at least 50 %, in particular by at least 75 %, in particular by at least 100 %, in particular by at least 150 %, in particular by at least 200 % in comparison to the combined value as established in a healthy reference population.

Preferably, in step c) the weighted combined value is compared to the corresponding weighted combined value of a reference population, wherein an increased weighted combined value of the female is indicative of an increased risk of having PCOS, particularly wherein the weighting factors have been or are obtained by analyzing a population of female’s having PCOS and/or a population of females without PCOS.

For example, the combined value obtained in step b) may further be processed by a weighting factor. This weighting factor may have been determined by analyzing a population of female’s having PCOS and/or a population of females without PCOS. For example, this may be the case, if the female assessed for having PCOS is known to be in a condition that may have similar effects in comparison to PCOS (e.g. , similar OA-value, HA-value, AMH-value) and which needs to be corrected for PCOS diagnosis.

Alternatively and preferably the analysis of a reference population may result in PCOS risk probabilities (see FIG. 3) associated to the health condition of a female, e.g. females being apparently healthy or having various forms or severities of PCOS allowing establishing cut-off values to differentiate the groups. Thresholds for the risk probabilities may be defined to allowing grouping of the females as likely having or not having PCOS. The group of females suffering from PCOS may be further subdivided according to the severity of PCOS, leading to more than two groups, such as no PCOS. PCOS with mild symptoms PCOS with severe symptoms, and allowing further differentiation. An exemplary procedure is described in the Examples,

In general, a threshold or cut-off represents an appropriate value to distinguish a female without PCOS from a female having PCOS. Further, if more than one threshold is applied, a threshold may represent an appropriate value to distinguish women without PCOS from females having various symptoms, forms or severities of PCOS.

A suitable threshold may be chosen depending on the sensitivity and specificity desired. Sensitivity and specificity are statistical measures of the performance of a binary classification test, also known in statistics as classification function

Sensitivity (also called the true positive rate) measures the proportion of positives that are correctly identified as such (e.g., the percentage of females having a symptom of PCOS or having PCOS who are correctly identified as having this disease).

Specificity (also called the true negative rate) measures the proportion of negatives that are correctly identified as such (e.g., the percentage of healthy females who are correctly identified as not having a symptom of PCOS or PCOS).

The threshold can be set in order to either increase sensitivity or specificity. A value above such a threshold may be considered as increased. Preferably, a threshold is chosen to match the diagnostic question of interest.

Further, if considered alone, all values of the dataset of step a) are only regarded to be indicators for a risk of having PCOS. Not each increment of a single value compared to the healthy reference population automatically may be considered as a confirmation of having PCOS. Finally, it is the overall processing of the data set provided in step a) according to step b) into a combined value and the comparison of the combined value obtained in step b) to the corresponding combined value as established in a reference population, which finally allow assessing a fernale’s risk of having PCOS.

Therefore, it is possible that single values of the dataset of step a) may be increased compared to the reference population, while at the same time others are not increased in comparison to the healthy reference population, but the female is not considered of having PCOS, e.g., because the significance of the increased value(s) is decreased by its / their weighting factor(s) and / or the significance of the value(s) not increased is increased by its / their weighting factor(s).

However, it may also be possible, that not all values of the dataset of step a) may be increased in comparison to the healthy reference population, while at the same time others are not increased in comparison to the reference population, and the female is considered of having PCOS, e.g., because the significance of the increased value(s) is increased by its / their weighting factor(s) and / or the significance of the value(s) not increased is decreased by its / their weighting factor(s).

Step d)according to the method of the first aspect of the invention, comprises indicating the female’s risk of having PCOS via an indication unit.

The term “indicating” describes showing or displaying of the female’s risk of having PCOS as assessed by the method according to the first aspect of the invention.

The risk may be indicated in various ways, such as by words, numbers, scales or colours or in any other way known to the person skilled in the art.

For example, the risk may be indicated by the simple display of words having the meaning of “yes” and “no”, or “PCOS” and “no PCOS” in any language,

The risk of having PCOS may also be displayed by the use of numbers, such as a low number (e.g. “0”) for no risk of having PCOS or a high number (e.g, “100” or “10”) for having the highest risk of having PCOS. Also, percentile numbers may be applicable, such as 0% and 100% Further the numbers may also display various degrees of the risk of having PCOS, whereby e.g. all numbers between the lowest and highest number on a continuous range may be suitable (e.g. all values between “0” and “100”). The numbers may further be displayed using a scale, such as used for a speedometer in a car.

Further, the risk may be displayed using colours for the different outcomes of the method according to the first aspect of the invention. For example, green may display a low risk of having PCOS, while red may display a high risk of having PCOS. Further, the colours may also display various degrees of the risk of having PCOS, such as green for a low risk, yellow for a moderate risk and red for a high risk, Moreover, the different degrees may be depicted by continuous colour transitions, e.g. from green via yellow to red. For each of these examples, colours are exchangeable.

For example, and considering the above exemplified X_{OA,} X_{HA} and X_{AMH}-values for humans, a risk score classification may be as follows:
High risk score (red): 6-9 points
Moderate risk score (yellow); 3-5 points
Low risk score (green): 0-2 points

Alternatively, a risk score classification may be chosen based on deciles of risks, e.g. as follows.
High risk score (red): 67% to 100%
Moderate risk score (yellow): 34% to 66%
Low risk score (green): 0% to 33%

Alternatively, a risk score classification may be determined by mathematically optimizing the thresholds for risk classification using prespecified threshoid(s) for sensitivity and/or specificity or risk deciles e.g. using a predictiveness curve (Pepe et al. 2008),

The term “indication unit” describes an electronic device or part of an electronic device displaying the results of the operations of the processing unit. Suitable indication units are well-known to the person skilled in the art. For example, the indication unit may be any kind of visual display integrated in an electronic device, such as a computer or mobile device, e.g. smartphone. Further, the indication unit may be any kind of visual display that can be connected to a computer or mobile device, e.g. smartphone, but is considered as a separate device.

For example, the method according to the first aspect of the inventionmay be easily performed during a visit to the doctor. During this visit, the female may be asked for the length of her menstrual cycle. Further, after taking a blood sample, the androgen level, such as the amount of total testosterone and SHBG. and the amount or concentration of AMH may be determined in laboratory tests. The physician may then easily enter the results (length of menstrual cycle (see definition above), androgen amount or concentration, such as the amount or concentration of total testosterone and SHBG or the FAI, and amount or concentration of AMH) in a computer program or application (app). In addition, the program may also be capable of calculating the FAI, if data for total Testosterone and SHBG are provided. The program or application may then, based on the method according to the first aspect of the invention, automatically determine the OA-, HA- and AMH-values, compare these values to the values of a reference population, consider potentially weighting factors and, as a result, indicate the female’s risk of having PCOS. This indication may simply be a number or any kind of colour code as described above, Thereby, the physician would only have to type in the numbers retrieved from the female and the laboratory into the program or application. No further estimation or analysis would be required. Further diagnoses depending on the results of the method of the first aspect of the invention may be described above, such as a diabetes test. Moreover, treatment may be immediately started, such as the administration or cessation of a contraception pill to regularize the female menstrual cycle.

Alternatively, the method of the first aspect may be performed partially by the patient and partially by a medical expert, such as a physician Exemplified, fine patient may enter her cycle length and or phenotypical symptoms of HA (e.g. skin or hair symptoms) into an app or software program. If these deviate from a healthy reference, the app or software program suggests the patient, to see a physician for further evaluation. During such visit and after laboratory testing of the androgen level and AMH value as described above, the physician may easily enter the results (androgen amount or concentration, such as the amount or concentration of total testosterone and SHBG or the FAI, and amount or concentration of AMH) into said a computer program or app. As described above, the software program or app may then, based on the method according to the first aspect of the invention, automatically determine the OA-, HA- and AMH-values, compare these values to the values of a healthy reference population, consider potentially weighting factors and, as a result, indicate the female’s risk of having PCOS.

In a preferred embodiment, the data set of step a) of the method of the first aspect of the invention further includes
an WEIGHT-value reflecting the female’s body weight, wherein an increased WEIGHT-value relative to the WEIGHT-value of a normal weight population indicates an increased body weight; and/or
an AGE-value reflecting the female’s age, wherein the AGE-value correlates age-dependent hormone levels.

Especially preferred, the data set of step a) of the method of the first aspect of the invention further includes an OA-value, a HA-value, an AMH-value and an AGE-value, even more preferably an OA-value, a HA-value, an AMH-value, an AGE-value and a WEIGHT-value.

The data set of step a) of the method of the first aspect of the invention may comprise additional values reflecting further conditions of the female’s body or health. Relevant factors and corresponding values suitable for indicating a female’s risk of having PCOS are well-known to a person skilled in the art. For example, the values may reflect a female’s body weight (WEIGHT-value) and / or age (AGE-value).

In a preferred embodiment, the data set of step a) of the method of the first aspect of the invention further includes a WEIGHT-value reflecting the female’s body weight, wherein an increased WEIGHT-value relative to the WEIGHT-value of a reference population indicates an increased body weight. An increased body weight is one possible symptom of PCOS.

The term “WEIGHT-value” describes a value reflecting the female’s deviation (increase) from the normal body weight (i.e., the weight of a corresponding normal weight female). An increased WEIGHT-value relative to the WEIGHT-value of a reference population indicates an abnormal weight, which is a symptom observed in some females having PCOS. Usually, the WEIGHT-value directly correlates with the body weight A female having a normal weight is referred to as symptom-free with respect to the WEIGHT-value. Overweight/obesity is one possible symptom of PCOS

The term “body weight” describes the female’s mass or weight Suitable methods for determining the female’s body weight are well-known to the person skilled in the art, such as personal scales. Usually, body weight is measured without any items (such as clothes, shoes, and accessories) located on the female. Body weight may vary throughout the day, as the amount of water in the body may not be constant, e.g. due to activities such as drinking, urinating, or exercise. Therefore, in case: of by statistical analysis, such as forming a median or mean value. Further suitable methods for statistical analysis are well-known to the person sₖilled in the art

The term “normal weight” describes the standard weight value of healthy,_{;} reproductive-aged females. The term “normal weight population” describes a population of healthy, non-obese, reproductive-aged females.

Increased weight is translated into an increased WEIGHT-value relative to the WEIGHT-value of females with normal weight. Therefore, an increased WEIGHT-value relative to the WEIGHT-value of a reference population is considered to indicate an increased body weight of the female, which is indicative of an increased risk of having PCOS.

Values defining normal weight, underweight and overweight/obesity are wall-known in the art. Values defining normal body weight may be obtained from standard publications or from a healthy reference population (see also above with respect to the OA-value).

For human females, the WEIGHT-value (X_{WEIGHT}) may for example be determined based on the female’s degree of overweight or obesity. It is possible to apply a continuous correlation, i.e. a high overweight or obesity may then correspond to a high WEIGHT-value, while a low overweight or obesity may correspond to a low WEIGHT-value.

Further, the value used for determining the WEIGHT-value, such as the degree of overweight or obesity, may be subject of any mathematical operation before the WEIGHT-value is determined. Suitable mathematical operations are well-known to the person skilled in the art and comprisefor example, addition, subtraction, muₗtiplication, division or logarithmising.

Moreover, the WEIGHT-value (X_{WEIGHT}) may for example be determined by grouping of values (e.g. by forming percentiles) and determining thresholds and / or out-off values. Thresholds, cut-off values, grouping, translation of values etc. may be defined as described above with respect to the OA-value.

The WEIGHT-value (X_{WEIGHT}) may for example be determined as follows:
X_{Weight} =2, if the female is overweight;
X_{weight} =0, if the female is normal weighted.

In addition to the normal weight of a female, the female’s body size or measurements of specific parts of the female’s body may be considered for calculating the WEIGHT-value, such as by including the BodyMass Index (BMI), the Broca-lndex, the Ponderal-Index, the Waist-Hip-ratio, the Waist-to-height ratio or the waist circumference. The body size of the female or measurements of specific parts of the female’s body may be measured by methods known to the person skilled in the art, such as by using a measuring stick or a tape measure. Exemplified, if the BMI is considered, women above 19 years of age are considered to be underweight with a BMI of below 19, are considered to be of normal weight with a BMI of 10-24, are considered to be overweight with a BMI of 25-29, and are considered to be obese with a BMI of above 30 (see e.g. tabelle.htm)

The WEIGHT-value of the female may be considered to be higher than the WEIGHT-value of the healthy reference population, if the BMI is at least 25_{:} preferably at least 26, more preferably at least 27, more preferably at least 28, and mostly preferred at least 29.

In a further preferred embodiment, the data set of step a) of the method of the first aspect of the invention further includes an AGE-value reflecting the female’s age..

The female’s age may also influence the other parameters determined. For example, hormone concentrations in a female may vary depending on her age, as shown for AMH in Table 1 above. Therefore, it may be an option to introduce an AGE-value to correct age-dependent variations of the other values, such as the AMH-value. In this manner, all other values or only certain values, such as the AMH-value, may be corrected.

The term “AGE-value” describes a value reflecting the female’s age. This may be easily retrieved by asking the female, Usually the AGE-value directly or indirectly correlates with the female’s age,

If in this preferred embodiment, a weighting factor is applied to each value (OA-value, HA-value and AHM-value, and AGE-value, and optional WEIGHT-value), the data set providedin step a) mayfor examplebe combined using the following algorithm (PCOS Scoring algorithm):

$$\begin{matrix}\text{Score} \text{=} {\text{X}}_{\text{OA}} * {\text{W}}_{\text{OA}} + {\text{X}}_{\text{HA}} * {\text{W}}_{\text{HA}} + {\text{X}}_{\text{AMH}} * {\text{W}}_{\text{AMH}} + {\text{X}}_{\text{AGE}} * {\text{W}}_{\text{AGE}} \text{or} \\ \text{Score} \text{=} {\text{X}}_{\text{OA}} * {\text{W}}_{\text{OA}} + {\text{X}}_{\text{HA}} * {\text{W}}_{\text{HA}} + {\text{X}}_{\text{AMH}} * {\text{W}}_{\text{AMH}} + {\text{X}}_{\text{AGE}} * {\text{W}}_{\text{AGE}} \text{+} \\ {\text{X}}_{\text{WEIGHT}} * {\text{W}}_{\text{WEIGHT} \text{.}}\end{matrix}$$

whereby X_{OA}, X_{HA}, X_{AMH}, X_{WEIGHT} and X_{AGE} may be defined as described above and W_{OA}, W_{HA}. W_{AMH}, W_{WEIGHT} and W_{AGE} are weighting factors for weighting X_{OA}, X_{HA}, X_{AMH}, X_{WEIGHT} and X_{AGE}.

To determine the Score, any interactions between X_{OA}, X_{HA}, X_{AMH}, _{WEIGHT}, X_{AGE} W_{OA}, W_{HA}, W_{AHM}, W_{WEIGHT} and / or W_{AGE} are possible. Interaction terms as mentioned above may also possible be included in in the formula.

Moreover, these interactions comprise any mathematical operation known to the person skilled in the art, such as addition, subtraction, multiplication, division or logarithmising.

In a preferred embodiment, the data set provided in step a) consists of the OA-value, the HA- value, the AMH- value, the WEIGHT-value and the AGE-value, optionally in combination with the PHE-vaiue, which are the only values processed in step b) to obtain the combined value.

In another preferred embodiment, the HA-velue of the data set provided in step a) of the method of the first aspect of the invention corresponds to
the amount or concentration of free testosterone (FT) in a sample obtained from the female, or
the ratio of the amount or concentration of total testosterone (TT> and the amount or concentration of sex hormone-binding globulin (SHBG) in a sample obtained from the female (TT/SH8G), optionally multiplied by a constant a (a *TT/SHBG), especially multiplied by 100 (100) * TT/SHBG).

In a preferred embodiment, the HA-value of the data set provided in step a) of the method of the first aspect of the invention corresponds to the amount or concentration of free testosterone (FT) in a sample obtained from the female.

The term “free testosterone” describes testosterone in the blood that is not attached to any protein. Exemplified for the Elecsys Testosterone assay, a woman being from 20 years to 49 years old has a bioavailable “free” testosterone values of 0.059-0.756 nmol/L.

Suitable methods for detecting the amount or concentration of free testosterone (FT) in a sample are, for example, mass spectrometry, such as liquid chromatography-mass spectrometry (LCMS) / mass spectrometry. For example, a needle may be used to draw blood from a vein in arm or hand.

If the HA-value of the data set provided in step a) of the method of the first aspect of the invention corresponds to the amount or concentration of free testosterone (FT) in a sample obtained from the female, the HA-value (X_{HA}) may for example be determined depending on thresholds.

For example, the X_{HA}-value corresponding to the amount or concentration of free testosterone (FT) in a sample for healthy females may be allocated 0, while all X_{HA} of females having a higher amount or concentration of free testosterone (FT) may be grouped and allocated another number, preferably 2.

If free testosterone is used for determining the HA-value, the HA-value (X_{HA}) may for example be determined as follows:
X_{HA} = 2, if the free testosterone > threshold (whereby the threshold may be e.g., 1.1 ng/dl, preferably 1.3 ng/dl, more preferably 1.5 ng/dl, mostly preferred 2.0 ng/dl);
X_{HA} = 0, if the female is symptom-free (i.e. does not show increased free testosterone)

In another preferred embodiment the HA-value of the data set provided in step a) of the method of the first aspect of the invention corresponds to the ratio of the amount or concentration of total testosterone (TT) and the amount or concentration of sex hormone-binding globulin (SHBG) in a sample obtained from the female (TT/SHBG), optionally multiplied by a constant a (a * TT/SHBG), especially multiplied by 100 (100 * TT/SHBG).

This preferred embodiment shows two options for determining a HA-value, which have already been explained above:
1) Total Testosterone / SHBG × 100, which corresponds to the calculation of an FAI as described above; and
2) (Total Testosterone / SHBG x 100)*a, which corresponds to the calculation of a weighted FAI that was described above as well. Thereby, constant a corresponds to W_{HA}.

In a further preferred embodiment, in the method according to the first aspect of the invention,
the female has a high risk, if the combined value is above a threshold_{high}; and
the female has a moderate risk, if the combined value is above a threshold_{moderate} and below threshold_{high}; and
the female has a low risk, if the combined value is below threshold_{moderate}.

Further, the risk of a female having PCOS may be determined using one or more thresholds or cut-off values. For determining these thresholds, the combined values of females belonging to a reference population, e.g. including 1) to the healthy reference population without PCOS and 2) having various degrees and forms of PCOS are determined. This may lead to various cut-off values or thresholds for healthy females and female’s having different risks/degrees for PCOS.

These combined values may then be grouped in e.g. three groups, preferably based on statistical methods, which are well-known to the person skilled in the art. In a next step, thresholds may be determined for each groups, e.g. the lowest or highest combined value of each group.

The combined values may be grouped in three or more groups, whereby each group represents a different risk of having PCOS. For example, the combined values are grouped into three groups of a low, moderate, and high risk of having PCOS.

The group representing the lowest risk of having PCOS may be up to the highest combined value below the group having a moderate risk of having PCOS (e.g. threshold_{moderate}). The group representing the highest risk of having PCOS may have a threshold comprising the highest combined value of the group representing the moderate risk of having PCOS (threshol_{high}). The female may be considered to have a high risk, if the combined value is above a threshold_{high}; and the female has a moderate risk, if the combined value is above or the same as a threshold_{moderate} and below threshold_{high}; and the female has a low risk, if the combined value is below threshold_{moderate}.

In a further preferred embodiment, one or more values of the (healthy) reference population and/or the combined value of the reference population and/or the weighting factors for weighted calculation are retrieved from a database. This database may be any kind of database comprising patient data concerning symptoms indicative for PCOS and are well-known to the person skilled in the art.

In a preferred embodiment of the first aspect of the invention, the data set of step a) further includes a PHE-value reflecting one or more phenotypical characteristics known to be indicative of PCOS, wherein an increased PHE-value relative to the PHE-value of a healthy reference population indicates the presence of one or more phenotypical characteristics known to be indicative of PCOS is reflected by an increased PHE-value, particularly wherein the phenotypical characteristic is polycystic ovarian morphology (PCOM) and/or clinical hyperandrogenism, more particularly acne, seborrhea, alopecia, and/or hirsutism.

Preferably, the dataset of step a) further includes one, two, three or four values of the group consisting of the PHE-value, whereby any combination of these values may be possible.

Preferably, the data set of step a) further includes a PHE-value reflecting one or more phenotypical characteristics known to be indicative of PCOS, wherein an increased PHE-value relative to the PHE-value of a healthy reference population indicates the presence of one or more phenotypical characteristics known to be indicative of PCOS is reflected by an increased PHE-value, particularly wherein the phenotypical characteristic is polycystic ovarian morphology (PCOM) and/or clinical hyperendrogenism, more particularly acne, seborrhea, alopecia, and/or hirsutism.

The term “PHE-value” reflects one or more phenotypical characteristics known to be indicative of PCOS. Usually the PHE-value directly or indirectly correlates with one or more phenotypical characteristics known to be indicative of PCOS.

The term “phenotypical characteristic” describes any feature of the phenotype of a female known to be indicative of PCOS. For example, these phenotypical characteristics comprise polycystic ovarian morphology (PCOM) and/or clinical hyperandrogenism, such as acne, seborrhea, alopecia, and/or hirsutism. Preferably, these phenotypical characteristics comprise polycystic ovarian morphology (PCOM) and/or clinical hyperandragenism, more preferably acne, seborrhea, alopecia, deepening of voice and/or hirsutism.

These phenotypical characteristics of clinical hyperandrogenism may be simply diagnosed by asking the female or are apparent after a short physical examination of the female’s body.

Usually, a reference population does not show any or not more than one of these phenotypical characteristics known to be indicative of PCOS.

Presence of the above phenotypical characteristics is translated into an increased PHE-value relative to the PHE-value of females with normal appearance. Therefore, an increased PHE-value relative to the PHE-value of a reference population is considered to indicate the presence of phenotypical characteristics in the female, which are indicative of an increased risk of having PCOS.

For human females, the PHE-value (X_{PHE}) may for example be determined based on the female’s phenotype. lt is possible to apply a continuous correlation, i.e. a phenotype showing many features of the phenotype of a female known to be indicative of PCOS may then correspond to a high PHE-value, while a phenotype showing hardly any features of the phenotype of a female known to be indicative of PCOS may correspond to a low PHE-value.

Further, the value used for determining the PHE-value, such as the number or type of phenotypes, may be subject of any mathematical operation before the PHE-value is determined. Suitable mathematical operations are well-known to the person skilled in the art and comprise, for example, addition, subtraction, multiplication, division or logarithmising.

Moreover, the PHE-value (X_{PHE}) may for example be determined by grouping of values (e.g. by forming percentiles) and determining thresholds and / or cut-off values. Thresholds, cut-off values, grouping, translation of values etc. may be defined as described above with respect to the OA-value.

For humans, the PHE-value (X_{PHE}) may, for example, be determined as follows:
X_{PHE} = 2, if the female suffers from at least one phenotypical characteristic known to be indicative for PCOS, preferably at least two phenotypical characteristics known to be indicative for PCOS, more preferably at least three phenotypical characteristics known to be indicative for PCOS and mostly preferred at least four phenotypical characteristics known to be indicative for PCOS;
X_{PHE} = 0, if the female is symptom-free, i.e. does not reflect any phenotypical characteristics known to be indicative of PCOS

Beside the accumulation of phenotypical characteristics, the PHE-value may also consider the severity of each of these phenotypical characteristics, e.g, the severity of acne the female suffers from.

Moreover, the PHE-value may be weighted when combined with the values of the data set provided in step a) of the first aspect of the invention as described above for the data set provided in step a).

Preferably, the data set of step a) further includes additional values allowing to exclude other diseases such as non-classical adrenal hyperplasia (NCAH), androgen secreting tumors, Cushing syndrome, thyroid disorders, or hyperprolactinemia.

Accordingly, to exclude non-classical adrenal hyperplasia (NCAH), values for 17alpha-Hydroxyprogesterone (17-OHP) may be included.

Accordingly, to exclude androgen secreting tumors, values for androstenedione and dehydroepiandrosterone sulphate (DHEAS) (e.g. determined using the Roche Elecsys androstenedione or DHEA-S assay, respectively) may be included.

Accordingly, to exclude Cushing syndrome, values for Cortisol (e.g, determined using the Roche Elecsys Cortisol II assay) may be included.

Accordingly, to exclude thyroid disorders, values for Thyroid Stimulating Hormone (TSH) (e.g. Roche Elecsys TSH) may be included.

Accordingly, to exclude hyperprolactinemia, values for Prolactin (e.g. determined using Roche Elecsys Prolactin II assay) may be included.

In another preferred embodiment, the method according to the first aspect of the invention further includes determining one or more values of the data set provided in step a), particularly one or more of the value(s) corresponding to the amount or concentration of one or more of the hormone(s), especially by measuring the amount or concentration of one or more of the honmone(s) in the female’s sample.

In a further preferred embodiment, the amount or concentration of one or more of the hormone(s) in the female’s sample is measured by an immunoassay and/or mass spectrometry.

Suitable methods for determining the amount or concentration of one or more hormone(s) are well-know to the person skilled in the art. For example, suitable methods for the detection of the amount or concentration of hormones are an immunoassay and / or mass spectrometry, such as liquid chromatography-mass spectrometry (LCMS) / mass spectrometry, enzyme-linked immunosorbent assay (ELISA), electrochemiluminescence-immunoassay (ECLIA) and extraction / chromatography immunoassays, preferably electrochemiluminescence-Immunoassay (ECLIA), such as Elecsys® from Roche. Further, if the amount or concentration of more than one hormone(s) is determined, such as of two, three, four or more hormones, these may be determined separately in different measurements or together in one or more measurements).

In a second aspect, the invention relates to a kit for use in a method of assessing a female’s risk of having PCOS, the kit comprising reagents required to specifically measure in a sample obtained from the female
(i) the amount or concentration of FT or (ii) the amount or concentration of TT and the amount or concentration SHBG;
the amount or concentration of AMH; and
optionally the amount or concentration of one or more further hormones indicative of PCOS, especially wherein the kit is for use in the method of the first aspect of the invention, especially in a method of the first aspect,
wherein the method further includes determining one or more values of the data set provided in step a), particularly one or more of the value(s) corresponding to the amount or concentration of one or more of the hormone(s), especially by measuring the the amount or concentration of one or more of the hormone(s) in the female’s sample and/or
wherein the amount or concentration of one or more of the hormone(s) in the female’s sample is measured by an immunoassay and/or mass spectrometry.

The kit for use in a method of assessing a female’s risk of having PCOS usually comprises reagents required to specifically measure in a sample obtained from the female
... (1) the amount or concentration of FT or (ii) the amount or concentration of TT and the amount or concentration SHBG;
the amount or concentration of AMH; and optionally the amount or concentration of one or more further hormones indicative of PCOS.

In the context of the kit of the present invention the term “reagent describes a substance or compound added to a sample allowing to display the amount or concentration of a specific component in the sample,

In the context of the kit of the present invention the term “specifically measure” means to detect the exact amount or concentration of a clearly defined molecule. For a specific measurement the sample obtained from a female may be incubated with the reagent under conditions appropriate for formation of a binding agent marker-complex. Such conditions need not be specified since such appropriate incubation conditions are well-known to the skilled artisan.

Preferably, the kit comprises reagents to specifically measure
(i) FT or (ii) TT and SHBG, and
AMH.

Also preferably, the kit comprises reagents to specifically measure
(i) FT or (ii) TT and SHBG, and
AMH

More preferably, the kit comprises reagents to specifically measure
TT and SHBG, and
AMH.

Further, more preferably, the kit comprises reagents to specifically measure
TT and SHBG, and
AMH

The kit may also comprise further reagents for specifically measuring other molecules,, such as estrogens, androgens , (other than FT. TT, TTISHBG (FAI) and SHBG), dehydroepiandrosterone (DHEA), dehydroepiandrosterone sulfate (DHEA-S), androstenedione and/or dihydrotestosterone (DHT), Further suitable reagents are well-known to the person skilled in the art.

In the context of the kit of the present invention the term “reagent” may describe a protein molecule (such as an antibody), a nucleic acid molecule (such as any form of deoxyribonucleic acid (DNA) or ribonucleic acid (RNA)) or another biochemical, organic or anorganic substance, which may interact with the molecule to be specifically measured in a sample.

Further, the reagent may be linked to a detectable reporter moiety or label such as an enzyme, dye, radionuclide, luminescent group, fluorescent group or biotin, or the like, such as a fluorescent marker that may be used for immunoassays analysis. Any reporter moiety or label could be used with the reagent of the kit according to the second aspect of the invention so long as the signal of such may be directly related or proportional to the quantity of binding agent remaining on the support after wash. The amount of an optional second binding agent that remains bound to the solid support may then be determined using a method appropriate for the specific detectable reporter moiety or label. For radioactive groups, scintillation counting or autoradiographic methods are generally appropriate. Antibody-enzyme conjugates can be prepared using a variety of coupling techniques (for review see, e.g., Scouten, W. H., Methods in Enzymology 135:30-65, 1987). Spectroscopic methods can be used to detect dyes (including, for example, colorimetric products of enzyme reactions), luminescent groups and fluorescent groups. Biotin can be detected using avidin or streptavidin, coupled to a different reporter group (commonly a radioactive or fluorescent group or an enzyme). Enzyme reporter groups can generally be detected by the addition of substrate (generally for a specific period of time), followed by spectroscopic, spectrophotometric or other analysis of the reaction products Standards and standard additions can be used to determine the level of antigen in a sample, using techniques well-known to the person skilled in the art.

The reagent may also be a substance that may additionally be capable of linking to the matrix of a column used for chromatography for purification and / or further analysis (such as mass spectrometry analysis). Moreover, the reagent may be linked to a testing strip.

Preferably, the reagent is an antibody. Suitable antibodies for measuring the amount or concentration of one of the molecules to be specifically measured in a sample obtained from the female mentioned above, are well-known to the person skilled in the art.

The term “antibody” may comprise polyclonal antibodies, monoclonal antibodies, fragments thereof such as F(ab′)2, and Fab fragments, as well as any naturally occurring or recombinantly produced binding partners, which are molecules that specifically bind one of the molecules to be specifically measured in a sample Any antibody fragment retaining the above criteria of a specific binding agent can be used. Antibodies are generated by state of the art procedures, e.g., as described in Tijssen 1990 In addition, the skilled artisan is well aware of methods based on immunosorbents that can be used for the specific isolation of antibodies. By these means the quality of polyclonal antibodies and hence their performance in immunoassays -can be enhanced (Tijssen 1990). Preferably, the antibody is a monoclonal antibody.

For the achievements as disclosed in the present invention polyclonal antibodies raised in e.g. goats may be used. However, clearly also polyclonal antibodies from different species, eg., rats, rabbits or guinea pigs, as well as monoclonal antibodies can be used. Since monoclonal antibodies can be produced in any amount required with constant properties, they represent ideal tools in development of an assay for clinical routine.

Preferably, the reagent may be used in an electrachemlluminescence-immunoassay, more preferably, the reagent is an antibody that may be used in an electrochemiluminescence-immunoassay.

Moreover, the kit may comprise more than one reagent, such as two different reagents, three different reagents, four different reagents or more different reagents, preferably two different reagents to interact with one molecule that is specifically measured in a sample. For example, if the molecule that is specifically measured is measured in an electrochemiluminescence-immunoassay, the kit may comprise two different antibodies binding to the same molecule to be measured. Preferably, the two different antibodies binding to the same molecule are not competing for the binding site at the molecule and bind this molecule at different positions. Further, both antibodies may be linked to different detectable reporter moieties or labels. The sample that may be examined using the kit according to the second aspect of the invention is usually obtained from the female as described above.

Suitable methods for measuring the amount or concentration of one or more hormone(s) in the female’s sample are well-known to the person skilled in the art or as described above, Preferred methods for measuring the amount or concentration of one or more hormone(s) in the female’s sample are immunoassay / and or mass-spectrometry, such as liquid chromatography-mass spectrometry (LCMS) / mass spectrometry, enzyme-linked immunosorbent assay (ELISA), electrochemiluminescence-immunoassay (ECLIA) and extraction / chromatography immunoassays, preferably electrochemiluminescence-immunoassay (ECLIA), such as Elecsys® from Roche.

In an example for an electrochemiluminescense-immunoassay based on a ruthenium complex and tripropylamine (TPA), the first antibody may be bound to biotin and the second antibody may be bound to a ruthenium complex. During an incubation step with the molecule to be measured, both antibodies may bind to the same molecule to be measured, forming a sandwich complex. After incubation, the sandwich complex may be brought in contact with immobilized streptavidin, such as streptavidin linked to microparticles, whereby the sandwich complex may bind to the streptavidin microparticles via an interaction between biotin and streptavidin. Afterwards, the microparticles linked to the sandwich complex may be brought into a measuring cell, where the microparticles may be immobilized by magnetically interacting with the surface of an electrode. After removing of unbound substances, voltage is applied to the electrode inducing the emission of chemiluminescence that may be detected with a photomultiplier.

In an example for an enzyme-linked immunosorbent assay (ELISA), the sample may be incubated in a microwell plate, whose wells are covered with a first antibody towards the molecule to be measured, In a next step, after incubating and washing, a second antibody towards the molecule to be measured and linked with biotin may be added. After further incubating and washing, streptavidin-horseradish peroxidase (HRP) may be added. After a final incubating and washing, the substrate tetramethylbenzidine (TMB) may be added to the sample. Finally, an acidic stopping solution may be added. Measurement may be performed by dual wavelength absorbance measurement at 450 nm and between 600 and 630 nm. The absorbance measured is usually directly proportional to the concentration of AMH in the samples. A set of AMH calibrators may be used to pint a calibration curve of absorbance versus AMH concentration. The AMH concentrations in the samples may then be calculated from this calibration curve.

The step of specifically measuring may also be carried out as follows: The sample may be contacted with a first reagent (which could be immobilized, e.g. on a solid phase) under conditions allowing the binding of the first reagent to the substance to be measured. Unbound reagents may be removed by a separation step (e.g. one or more washing steps). A second reagent (e.g. a labeled agent) may be added to detect the bound first reagent to allow binding to and quantification of the same. Unbound second reagent may be removed. The amount of the second reagent which is proportional to the amount of the substance to be measured may be quantified, e.g. based on the label. Quantification may be done based on e.g. a calibration curve constructed for each assay by plotting measured value versus the concentration for each calibrator. The concentration or amount of the substance to be identified in the sample may be then read from the calibration curve.

Preferably, the kit for use in the method of the first aspect of the invention is further characterized, wherein the method further includes determining one or more values of the data set provided in step a), particularly one or more of the value(s) corresponding to the amount or concentration of one or more of the hormone(s), especially by measuring the the amount or concentration of one or more of the hormone(s) in the female’s sample and/or, wherein the amount or concentration of one or more of the hormone(s) in the female’s sample is measured by an immunoassay and/or mass spectrometry.

The kit may further comprise buffering agents and/or salts to adjust the pH as well as the reaction and measuring conditions. Moreover, the kit may comprise stabilizers, e.g to support the stability of the reagents and/or hormones during the specific measurement of (i) the amount or concentration of FT or (ii) the amount or concentration of TT and the amount or concentration SHBG: the amount or concentration of AMH; and the amount or concentration of one or more further hormones indicative of PCOS. Suitable buffers, salts and stabilizers are well-known to the person skilled in the art. In addition, sodium azide may be added to all liquid solutions of the kit, such as reagents or buffers.

The kit may also comprise all equipment necessary to take a blood sample from a female, such as a container for the blood sample, a needle and a device connecting the container and the needle. Preferably, the kit may comprise a syringe.

In general, a physician or a physician’s assistant may take blood from a female. Subsequently, the blood may be sent to a laboratory, where the sample is measured using the kit on a designated analyser, and the data are sent to the physician. However, the kit may also be applied by a physician or by a physician’s assistant himself. The kit may be applied during ambulatory, stationary treatment or domiciliary visit of the physician.

All components of the kit may be packaged separately in individual containers. However, it is also possible that two or more components of the kit may be packaged together in one or more containers.

The kit may further comprise a label, e.g. comprising instructions on how to use the kit or describing the kit’s contents. However, this information may also be provided in any other form, such as on storage media such as a CD-ROM or a USB stick.

In a third aspect the invention relates to the use of a marker combination comprising
(i) FT or TT/SHBG and
(ii) AMH
in combination with an OA-value, and optionally an WEIGHT-value and/or an AGE-value, in the assessment of the female’s risk of having PCOS, wherein the female has an increased risk of PCOS, when a combined value of the amount or concentration or ratio of the markers and the OA-value is increased relative to the combined value as established in a reference population, optionally wherein the marker combination further includes one or more further hormones indicative of PCOS

The term “marker” describes a clinical or biologic characteristic that is objectively measurable and that provides information on the risk of having or developing PCOS. A marker may indicate how well the patient is likely to do during the course of PCOS and PCOS treatment. A marker may further aim to objectively evaluate the patient’s overall outcome. Typically, markers are measured and evaluated at the time of diagnosis. The presence or absence of a marker can be useful for the selection of a female for treatment.

Suitable markers for detecting the risk of a female having PCOS are usually e.g., estrogens and/or androgens (such as testosterone (e.g. FT, TT, TT/SHBG (FAI), SHBG and / or bioavailable testosterone), dehydroepiandrosterone (DHEA), dehydroepiandrosterone sulfate (DHEA-S), androstenedione, and / or dihydrotestosterone (DHT)). Further suitable markers for detecting the risk of a female having PCOS are well-known to the person skilled in the art.

Preferably, markers for detecting the risk of a female having PCOS are
(i) FT or TT/SHBG and
(ii) AMH
in combination with an OA-value.

Also preferably, markers for detecting the risk of a female having PCOS are
(i) FT or TT/SHBG and
(ii) AMH
in combination with an OA-value, a WEIGHT-value and/or an AGE-value.

Further preferred, markers for detecting the risk of a female having PCOS are
(i) FT or TT/SHBG and
(ii) AMH
in combination with an OA-value; wherein the marker combination further includes one or more further hormones indicative of PCOS.

Further preferred, markers for detecting the risk of a female having PCOS are
(i) FT or TT/SHBG and
(ii) AMH
in combination with an OA-value, and a WEIGHT-value and/or an AGE-value; wherein the marker combination further includes one or more further hormones indicative of PCOS.

Further hormones indicative of PCOS are listed above as suitable markers for detecting the risk of a female having PCOS. These may comprise e.g., estrogens, androgens (other than FT, TT, TT/SHBG (FAI) and SHBG), dehydroepiandrosterone (DHEA), dehydroepiandrosterone sulfate (DHEA-S), androstenedione, and / or dihydrotestosterone (DHT)).

Further, the female may have an increased risk of PCOS, when a combined value of the amount or concentration or ratio of the markers and the OA-value is increased relative to the combined value as established in a reference population.

The combined value of the amount or concentration or ratio of the markers and the OA-value may be processed as described above for the processing of combined values.

The use of a marker combination of the third aspect of the invention may allow to determine a female’s risk of having PCOS. In particular, the female may have an increased risk of PCOS, when a combined value of the amount or concentration or ratio of the markers, as defined above, and the OA-value is increased relative to the combined value as established in a healthy reference population.

The combined value of the amount or concentration or ratio of the markers and the OA-value of a female is compared to the combined value of the amount or concentration or ratio of the markers and the OA-value of the reference population. If the the combined value of the amount or concentration or ratio of the markers and the OA-value of the female is increased in comparison to the the combined value of the amount or concentration or ratio of the markers and the OA-value of the healthy reference population, this may be an indicator for an increased risk of having PCOS of the female.

In general, the combined value of the amount or concentration or ratio of the markers and the OA-value of a female directly or indirectly correlates with her risk of having PCOS. Usually, the more the combined value of the amount or concentration or ratio of the markers and the OA-value of a female is increased in comparison to the combined value of the amount or concentration or ratio of the markers and the OA-value of the healthy reference population, the more increased is the risk of having PCOS of this female.

The combined value of the amount or concentration or ratio of the markers and the OA-value of the female may be considered to be higher than the combined value of the amount or concentration or ratio of the markers and the OA-value of the healthy reference population, if it is significantly higher than the combined value of the amount or concentration or ratio of the markers and the OA-value of the healthy reference population. Statistical procedures to assess whether two values are significantly different from each other are well-known to the person skilled in the art, such as Student’s t-test or chi-squared test.

In a fourth aspect, the invention relates to a computer system for use in a method of the first aspect, wherein the computer system comprises:
a) a data set unit comprising computer instructions for providing a data set including
   an OA-value reflecting the length of the female’s menstrual cycle and/or the number of the female’s menstrual cycles per year, wherein an increased OA-value relative to the OA-value of a healthy reference population indicates an abnormal menstrual cycle length and/or number;
   a HA-value reflecting the female’s androgen status, wherein an increased HA-value relative to the HA-value of a healthy reference population indicates an increased androgen level in the female; and
   an AMH-value corresponding to the amount or concentration of AMH in a sample obtained from the female;
b) a processing unit comprising computer instructions for processing the data sets of step a), wherein the processing comprises combining values of the data set provided in step a) into one combined value;
c) a reference data unit comprising computer instructions for
   (i) storing and/or retrieving a reference data set including one or more of the reference values as established in a reference population and processing the reference data set into a combined value of the reference population; or
   (ii) storing and/or retrieving a combined value of the reference population;
(d) comparing the combined value obtained in step b) to the corresponding combined value of step c), wherein an increased combined value of the female relative to the combined value of the reference population is indicative of an increased risk of PCOS; and
e) an indication unit indicating the female’s risk of having PCOS.

The term “computer instructions” such as used in segments a), b) and c) of to the fourth aspect of the invention describes a set of machine language instructions that a particular processor may understand and execute.

Further, according to segment c) of the computer system according to the fourth aspect of the invention, a reference data unit comprising computer instructions for
(i) storing and/or retrieving a reference data set including one or more of the reference values as established in a reference population and processing the reference data set into a combined value of the reference population; or
(ii) storing and/or retrieving a combined value of the reference population.

The term “reference data set” describes a collection of reference values including one or more of the reference values as established in a reference population such as in a reference population including females without PCOS and females having any form of PCOS, preferably as established in a healthy reference population without PCOS. This reference data set may further be processed into a combined value of the reference population or in a combined value of a reference population comprising healthy females without PCOS and females having any form of PCOS, preferably into a combined value of the reference population. The processing into a combined value may be performed as described above.

Further features of this fourth aspect of the present invention are described above or are well-known to the person skilled in the art.

In a preferred embodiment, the computer system according to the fourth aspect of the invention is further characterized as in any embodiments of the first aspect of the invention.

In a fifth aspect, the invention relates to computer program comprising instructions which, when the program is executed by a computer, cause the computer to carry out steps a), b), c) and d) of any of the methods of the first aspect.

The computer program may cause a computer to perform the method according to the first aspect of the invention as above when executed.

The computer program may be directly loadable into the internal memory of a digital computer, may comprise software code portions suitable for implementing the method according to the first aspect of the invention when said product is run on a computer

The computer program may be a computer program preferably stored on a machine readable storage medium like RAM, ROM, or on a removable and/or portable storage medium, such as, but not limited to a CD-ROM, flash memory, DVD, BlueRay, FlashDisk, a storage card or a USB-stick, The computer program may be provided on a server to be downloaded via for example a data network such as the internet or another transfer system such as a phone line or a wireless transfer connection. Additionally, or alternatively, the computer program may be a network of computer implemented computer programs such as on a client/server system or a cloud computing system, an embedded system with a computer program or on an electronic device, like a smart phone or a personal computer on which computer programs are stored, loaded, run, exercised, or developed.

In a sixth aspect, the invention relates to a computer-readable storage medium comprising instructions which, when executed by a computer, cause the computer to carry out steps a), b), c) and d) of any of the methods of the first aspect.

The term “computer-readable storage medium” describes any computer readable medium capable of storing data, such as executable code.

The products and uses according to the second, third, fourth and fifth aspect may be further defined as specified for the method of the first aspect of the present invention, Particularly, with respect to the terms used in the second, third, fourth and fifth aspect of the present disclosure it is referred to the terms, examples and specific embodiments used in the first aspect of the present disclosure, which are also applicable to the other aspects of the present disclosure.

In further embodiments, the present invention relates to the following aspects:

1. A method of assessing a female’s risk of having polycystic ovary syndrome (PCOS), the method comprising
a) providing a data set including
   an OA-value reflecting the length of the female’s menstrual cycle and/or the number of the female’s menstrual cycles per year, wherein an increased OA-value relative to the OA-value of a healthy reference population indicates an abnormal menstrual cycle length and/or number,
   a HA-value reflecting the female’s androgen status, wherein an increased HA-value relative to the HA-value of a healthy reference population indicates an increased androgen level in the female, and
   an AMH-vaiue corresponding to the amount or concentration of anti-Müllerian hormone (AMH) in a sample obtained from the female;
b) processing the data set provided in step a) with a processing unit, wherein the processing comprises combining values of the data set provided in step a) into one combined value;
c) comparing the combined value obtained in step b) to the corresponding combined value as established in a reference population, wherein an increased combined value of the female relative to the combined value of a healthy reference population is indicative of an increased risk of PCOS; and
d) indicating the female’s risk of having PCOS via an indication unit.

2. The method of aspect 1, wherein the data set of step a) further includes
a WEIGHT-value reflecting the female’s body weight, wherein an increased WEIGHT-value relative to the WEIGHT-value of a normal weight population indicates an increased body weight; and/or
an AGE-value reflecting the female’s age.

3. The method of aspect 1 or 2, wherein the HA-value corresponds to
the amount or concentration of free testosterone (FT) in a sample obtained from the female, or
the ratio of the amount or concentration of total testosterone (TT) and the amount or concentration of sex hormone-binding globulin (SHBG) in a sample obtained from the female (TT/SHBG), optionally multiplied by a constant a (a * TT/SHBG), especially multiplied by 100 (100 * TT/SHBG).

4. The method of any of aspects 1 to 3, wherein in step b) the combined value is a weighted combined value obtained by weighted calculation of the values provided in step a) and in step c) the weighted combined value is compared to the corresponding weighted combined value of a reference population, wherein an increased weighted combined value of the female is indicative of an increased risk of having PCOS, particularly wherein the weighting factors have been or are obtained by analyzing a reference population comprising healthy females and/or female’s diagnosed with PCOS.

5. The method of any of aspects 1 to 4, wherein
the female has a high risk, if the combined value is above a threshold_{high}; and
the female has a moderate risk, if the combined value is above a threshold_{moderate} and below threshold_{high}; and
the female has a low risk, if the combined value is below threshold_{moderate}.

6. The method of any of aspects 1 to 5, wherein one or more values of the reference population and/or the combined value of the reference population and/or the weighting factors for weighted calculation are retrieved from a database.

7. The method of any of aspects 1 to 6, wherein the data set of step a) further includes PHE-value reflecting one or more phenotypical characteristics known to be indicative of PCOS, wherein an increased PHE-value relative to the PHE-value of a healthy reference population indicates the presence of one or more phenotypical characteristics known to be indicative of PCOS is reflected by an increased PHE-value, particularly wherein the phenotypical characteristic is polycystic ovarian morphology (PCOM) and/or hyperandrogenemia, more particularly acne, seborrhea, alopecia, and/or hirsutism.

8. The method of any of aspects 1 to 7, wherein the sample is a blood sample, particularly selected from the group consisting of serum, plasma, and whole blood,

9. The method of any of aspects 1 to 8, wherein the female is a human.

10. The method of any of aspects 1 to 9, wherein the method further includes determining one or more values of the data set provided in step a), particularly one or more of the value(s) corresponding to the amount or concentration of one or more of the hormone(s), especially by measuring the amount or concentration of one or more of the hormone(s) in the female’s sample.

11. The method of aspect 1 to 10, wherein the amount or concentration of one or more of the hormone(s) in the female’s sample is measured by an immunoassay and/or mass spectrometry.

12. A kit for use in a method of assessing a female’s risk of having PCOS, the kit comprising reagents required to specifically measure in a sample obtained from the female
(i) the amount or concentration of FT or (ii) the amount or concentration of TT and the amount or concentration SHBG;
the amount or concentration of AMH; and
optionally the amount or concentration of one or more further hormones indicative of PCOS,
especially wherein the kit is for use in the method of aspect 10 or 11.

13. Use of a marker combination comprising
(i) FT orTT/SHBG and
(ii) AMH
in combination with an OA-value, and optionally a WEIGHT-value and/or an AGE-value, in the assessment of the female’s risk of having PCOS, wherein the female has an increased risk of PCOS, when a combined value of the amount or concentration or ratio of the markers and the OA-value is increased relative to the combined value as established in a reference population, optionally wherein the marker combination further includes one or more further hormones indicative of PCOS,

14. A computer system for use in a method of any of aspects 1 to 11, the computer system comprising:
a) a data set unit comprising computer instructions for providing a data set including
   an OA-value reflecting the length of the female’s menstrual cycle and/or the number of the female’s menstrual cycles per year, wherein an increased OA-value relative to the OA-value of a healthy reference population indicates an abnormal menstrual cycle length and/or number:
   a HA-value reflecting the female’s androgen status, wherein an increased HA-value relative to the HA-value of a healthy reference population indicates an increased androgen level in the female; and
   an AMH-value corresponding to the amount or concentration of AMH in a sample obtained from the female;
b) a processing unit comprising computer instructions for processing the data sets of step a), wherein the processing comprises combining values of the data set provided in step a) into one combined value;
c) a reference data unit comprising computer instructions for
   (i) storing and/or retrieving a reference data set including one or more of the reference values as established in a reference population and processing the reference data set into a combined value of the reference population; or
   (ii) storing and/or retrieving a combined value of the reference population:
(d) comparing the combined value obtained in step b) to the corresponding combined value of step c), wherein an increased combined value of the female relative to the combined value of a healthy reference population is indicative of an increased risk of PCOS; and
e) an indication unit indicating the female’s risk of having PCOS.

15. The computer system of aspect 14, further characterized as in any of aspects 2 to 11,

16. Computer program comprising instructions which, when the program is executed by a computer, cause the computer to carry out steps a), b), c) and d) of any of the methods of aspects 1 to 11.

17. Computer-readable storage medium comprising instructions which, when executed by a computer, cause the computer to carry out steps a), b), c) and d) of any of the methods of aspects 1 to 11.

In general, the disclosure is not limited to the particular methodology, protocols, and reagents described herein because they may vary. Further, the terminology used herein is for the purpose of describing particular embodiments only and is not intended to limit the scope of the present disclosure. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the disclosure. Although any methods and materials similar or equivalent to those described herein can be used in the practice as presented herein, the specific methods, and materials are described herein.

The disclosure is further illustrated by the following figures and examples, although it will be understood that the figures and examples are included merely for purposes of illustration and are not intended to limit the scope of the disclosure unless otherwise specifically indicated.

### FIGURES

FIG. 1 illustrates ROC curves resulting from weighted logistic regression evaluated using 200 Monte-Carlo cross-validation runs for 1955 cases and 1642 controls based on all variables of the PCOS risk score (age, BMI, OA, FAI, and AMH; Area under the curve (AUC):0.976) compared to single variables OA (AUC:0.898), FAI (AUC:0.765) or AMH (AUC:0.838) alone.

FIG. 2 shows ROC curves resulting from weighted logistic regression evaluated using 200 Monte-Carlo cross-validation runs for 1955 cases and 1642 controls based on all variables of the PCOS risk score (age, BMI, OA, FAI, and AMH; AUC:0.976) and combinations of AMH and FAI (AUC:0.877) or AMH and SHBG (AUC:0.873).

FIG. 3 illustrates a histogram of PCOS risk probabilities resulting from weighted logistic regression using age and BMI as well as OA, FAI and AMH using 1955 cases and 1642 controls. Crosses and circles indicate control and case subjects’ risks, respectively. Vertical lines denote PCOS risk classification derived using the predictiveness curve for 80% sensitivity and specificity.

FIG. 4 shows the Mean Regression coefficients (and mean SDs) of the weighted logistic regression model resulting from 200 Monte-Carlo cross-validation runs using 1955 cases and 1642 controls and the variables age and BMI (A) or age (B) as well as OA, FAI and AM H.

FIG. 5 depicts the Predictiveness curve of the PCOS risk score for 1955 cases and 1642 controls based on variables age, BMI, OA, FAI, and AMH (A) or variables age, OA, FAI, and AMH (B).

FIG. 6 shows ROC curves for prediction of case-control status of the independent second sample set consisting of 200 cases and 44 controls for the PCOS risk score and single variables of the PCOS risk alone. It visualizes the performance of the PCOS risk score based on 44 controls and 200 cases described in Example 3. The ROC curves as well as the AUC demonstrated that the combined variables of the PCOS risk score (age, BMI, OA, FAI and AMH) are superior as to using the single variables OA, FAI or AMH alone. The AUC of the PCOS risk score was 0.99 indicating a very good separation between cases and controls, followed by OA (AUC: 0.96), AMH (AUC: 0.76), and FAI (AUC:0.90). The performance of the PCOS risk score for the four-variable combinations (variables OA, FAI, AMH, and AGE of the PCOS risk) was also assessed (see Table 9).

FIG. 7 shows ROC curves for prediction of case-control status of the second sample set consisting of 200 cases and 44 controls for the PCOS risk score with the combined variables age, BMI, OA, FAI, AMH and combinations of AMH + FAI and AMH + SHBG. It illustrates the performance of the PCOS risk score including all variables (age, BMI, OA, FAI, and AMH) compared to a combination of AMH and FAI or a combination of AMH and SHBG (as suggested by Calzada et al.). The highest AUC was found for the PCOS risk score (AUC:0.99). An AUC of 0.90 was for the combination of AMH + FAI (AUC:0.90) and the combination of AMH +SHBG (AUC:0.90). The ROC curves as well as the AUC show that the combined variables of the PCOS Risk score (age, BMI, OA, FAI, and AMH; AUC: 0.96) are superior to using AMH combined with either FAI (AUC: 0.90) or AMH and SHBG (AUC:0.90)

FIG. 8 shows ROC curves for prediction of case-control status of the second sample set consisting of 200 cases and 44 controls for the biochemical detection of Hyperandrogenism (HA). FAI was compared to LH, FSH and the LH/FSH ratio. The highest AUC was found for FAI (AUC: 0.90) followed by the ratio of LH/FSH (AUC: 0.85).

FIG. 9 illustrates a histogram of PCOS risk probabilities for the second sample set consisting of 200 cases and 44 controls The weights of the PCOS risk were derived using weighted logistic regression with 200 MCCV runs and 100 repetitions using age. BMI, OA. FAI and AMH in the sample set of 1866 cases and 1675 controls. The vertical lines indicate thresholds at low=0.2 and high=0.8 resulting in 96.0% sensitivity and 90.9% specificity. Crosses and circles indicate control and case subjects’ risks, respectively.

### EXAMPLES

### Example 1: Derivation of a PCOS Risk Score

In total, N = 1642 controls and N = 1955 cases were used for the derivation of the PCOS risk score based on combination of the numeric variables for age, BMI, FAI, and AMH as well as the categorical variable for Oligo Anovulation (OA) (yes, no). Additionally, the PCOS risk score was derived using a four-variable combination namely OA, age, FAI, and AMH but excluding BMI as well as using a three-variable combination namely OA, FAI, and AMH but excluding BMI and age.

### Cases

1955 women diagnosed with PCOS aged 20 to 45 years and not using contraceptives were considered OA was based on information of irregular menstrual cycles and/ or cycle length, Hyperandrogenism (HA) was derived as the free androgen index (FAI) based on levels of serum testosterone (nmol/l) and serum sex hormone-binding globulin (SHBG) (nmol/l):

$$\text{FAI} \text{=} \text{testosterone} \text{/} \text{SHBG * 100}$$

Patients were evaluated for PCOM by an ovarian volume ≥10 ml, and/or an antral follicle count (AFC) above threshold.

In addition, serum Anti-Müllerian Hormone (AMH) was measured using Elecsys AMH Plus immunoassay.

The PCOS cases covered PCOS patients representing the four phenotypes according to the Rotterdam criteria (PCOS Consensus Workshop Group, Fertil Steril 2004; 81:19-25).
Phenotype A (Oligo Anovulation (OA) +, Hyper-Androgenism (HA) +, polycystic ovarian morphology (PCOM) +)
Phenotype B (OA +, HA +, PCOM -)
Phenotype C (OA -, HA +, PCOM +)
Phenotype D (OA +, HA -, PCOM +)

### Controls

In the derivation of the PCOS risk score, the control group consisted of 1642 healthy women between 20 and 45 years without having PCOS.

Age and body mass index (BMI), antral follicle count (AFC) and serum AMH values were available, Testosterone and SHBG levels to depict the FAI were simulated based on information gained from the cases as well as the reference range values for healthy women to reflect the expected distribution of these variables in healthy subjects based on reference range studies. The simulation was done by sampling from the expected SHBG and Testosterone distributions in healthy women.

The following table lists the statistics of the variables for cases and controls:

TABLE 2

Descriptive statistics of the variables of the PCOS risk score overall and for patients (cases) and controls. For the controls, the FAI was derived based on simulated values for Testosterone and SHBG. OA of controls was based on information of the cycle length or simulated. The control group represented 45.6% of the overall number of patients and controls.

| | | All (N=3597) | Controls (N=1642) | Cases (N=1955) |
|---|---|---|---|---|
| OA* | Regular Cycle | N=1394 (38.8%) | N=1345 (81.9%) | N=49 (2.5%) |
| | OA | N=2203 (61.2%) | N=297 (18.1%) | N=1906 (97.5%) |
| FAI* | Mean | 4.6 | 2.7 | 6.1 |
| | SD | 4.5 | 2.2 | 5.3 |
| | Median | 3.2 | 2.1 | 4.9 |
| | Q1 ... Q3 | 1.8 ... 5.8 | 1.3 ... 3.4 | 2.6 ... 7.6 |
| | Min... Max | 0.20 ... 67.1 | 0.20 ... 27.2 | 0.28 ... 67.1 |
| AMH (ng/ml) | Mean | 4.9 | 2.7 | 6.7 |
| | SD | 4.0 | 2.3 | 4.2 |
| | Median | 3.9 | 2.2 | 5.6 |
| | Q1 ... Q3 | 2.1 ... 6.5 | 1.1 ... 3.6 | 3.8 ... 8.7 |
| | Min... Max | 0.010 ... 23.0 | 0.010 ... 22.1 | 0.010 ... 23.0 |
| Age (years) | Mean | 30.8 | 33.9 | 28.2 |
| | SD | 5.6 | 5.1 | 4.5 |
| | Median | 30.0 | 34.0 | 28.0 |
| | Q1 ... Q3 | 27.0 ... 35.0 | 30.0 ... 38.0 | 25.0 ... 31.0 |
| | Min ... Max | 20.0... 44.0 | 20.0 ... 44.0 | 20.0 ... 44.0 |
| BMI | Mean | 26.1 | 25.1 | 26.9 |
| | SD | 5.7 | 5.0 | 6.2 |
| | Median | 24.7 | 23.8 | 25.9 |
| | Q1 ... Q3 | 21.8 ... 29.5 | 21.6 ... 27.7 | 22.1 ... 30.7 |
| | Min ... Max | 13.9... 52.7 | 14.8 ... 52.7 | 13.9 ... 51.2 |
| Testosterone (nmol/L)* | Mean | 1.7 | 1.4 | 2.0 |
| | SD | 0.92 | 0.78 | 0.97 |
| | Median | 1.5 | 1.2 | 1.7 |
| | Q1 ... Q3 | 1.0 ... 2.2 | 0.89 ... 1.8 | 1.2 ... 2.5 |
| | Min ... Max | 0.17 ... 7.3 | 0.23 ... 6.2 | 0.17 ... 7.3 |
| SHBG (nmol/L)* | Mean | 56.0 | 69.5 | 44.6 |
| | SD | 35.8 | 40.7 | 26.3 |
| | Median | 47.8 | 59.7 | 38.3 |
| | Q1 ... Q3 | 31.5 ... 70.5 | 42.2 ... 86.6 | 26.2 ... 57.2 |
| | Min ... Max | 7.1 ... 370 | 8.8 ... 37.0 | 17.1 ... 342 |

*partially based on simulations.
OA: Oligomenorrhea and/or anovulation
FAI: Free Androgen Index; FAI = Testosterone / SHBG *100
BMI: Body Mass Index; BMI = weight (kg) / size (m)2.
AMH: Anti-Mullerian Hormone
SHBG: Sex Hormone-Binding Globulin

Of note, there was a difference in age between cases and controls due to the design of the study.

### PCOS Risk Score

The proposed PCOS risk score calculates the patient’s risk of having PCOS ranging from 0 to 1, with higher values meaning a higher risk of having PCOS.

$$\begin{matrix}\text{risk} \text{=} log \left(\frac{p}{1 - p}\right) = \text{log} \left(\frac{P \left(y = 1\right)}{1 - P \left(y = 1\right)}\right) \\ \begin{matrix}= {W}_{0} + {X}_{a g e} ∗ {W}_{a g e} \\ + {X}_{B M I} ∗ {W}_{B M I} \left(o p t i o n a l\right) \\ + {X}_{O A} ∗ {W}_{O A} \\ + {X}_{P A I} ∗ {W}_{P A I} \\ + {X}_{A M H} ∗ {W}_{A M H} ∈ \left[0 ; 1\right]\end{matrix}\end{matrix}$$

$$\begin{matrix}\text{with} \\ P \left(y = 1\right) = \text{probablitity of being a case,} \\ {\text{W}}_{0} = \text{intercept of the weighted logistic regression model,} \\ \text{variables} {X}_{a g e} = \text{age in years,} \\ {X}_{B M I} = \text{BMI} \left(\text{optional}\right) . \\ {X}_{O A} = \text{Oligo Anovulation} \left(\text{Regular/OA}\right) \\ {X}_{F A I} = \text{FAI} \text{.} \\ {X}_{A M H} = \text{AMH} \text{in ug/mL and} \\ \text{weights} {W}_{a g e} \text{for age} \text{.} \\ {W}_{B M I} \text{for BMI} \left(\text{optional}\right) . \\ {W}_{O A} \text{for OA} \text{.} \\ {W}_{F A I} \text{for FAI and} \\ {W}_{A M H} \text{for AMH} \text{.}\end{matrix}$$

Weighted logistic regression model was established with case-control status as endpoint within a Monte-Carlo cross-validation (MCCV) with 200 runs (Xu & Liang 2001).

The variables Age, BMI (optional), and OA as well as FAI were included for the derivation of the PCOS risk, whereas the variables AMH, testosterone and SHBG were included as log-transformed variables. To account for imbalance between the higher numbers of cases versus controls a weighted logistic regression model was applied to derive the PCOS risk. This means each subject was assigned a weight, which was considered within the model estimation of the logistic regression (Hastie et al. (2009). The weights were chosen according to Elkan (2001) by applying costs for the false classification of cases and controls each.

MCCV. For each of the MCCV runs the data set was randomly split into training and test set (80% and 20%, respectively), while maintaining the ratio of cases and controls. On the current training set, the model was built and the performance was evaluated by means of area under the ROC curve (AUC) using the respective test set. The estimated overall performance of the logistic regression model was given as mean AUC. Mean sensitivity and specificity was calculated to estimate the model performance for cases and controls, separately. The stability of the regression model was evaluated by providing the mean of the regression coefficients together with the standard deviation (SD) and coefficient of variation (CV).

The mean regression coefficients for each variable from MCCV were considered as the weights W̅ the PCOS risk.

The PCOS risk, i.e. the probability of being with PCOS (being a case), was than estimated by:

$$\text{risk} \text{=} \hat{p} = \hat{P} \left(y = 1\right) = \frac{exp \left({\hat{W}}_{0} + X \hat{W}\right)}{1 + exp \left({\hat{W}}_{0} + X \hat{W}\right)} ,$$

$$\begin{matrix}\text{with} \\ {\hat{W}}_{0} = \text{estimated intercept of logistic regression model,} \\ \hat{W} \text{estimated weights for age, BMI} \left(\text{optional}\right) , \text{OA, FAI and AMH and}\end{matrix}$$

$$\begin{matrix}X \hat{W} = {X}_{a g e} ∗ {\hat{W}}_{a g e} + {X}_{B M I} ∗ {\hat{W}}_{B M I} + {X}_{O A} ∗ {\hat{W}}_{O A} + {X}_{P A I} ∗ {\hat{W}}_{F A I} \\ + {X}_{A M H} ∗ {\hat{W}}_{A M H} .\end{matrix}$$

The PCOS risk classifies as low, moderate and high as follows: The risk thresholds were derived using the predictiveness curve as proposed by Pepe et al. (2007) given that at least 80% sensitivity and 80% specificity are achieved.

### Results

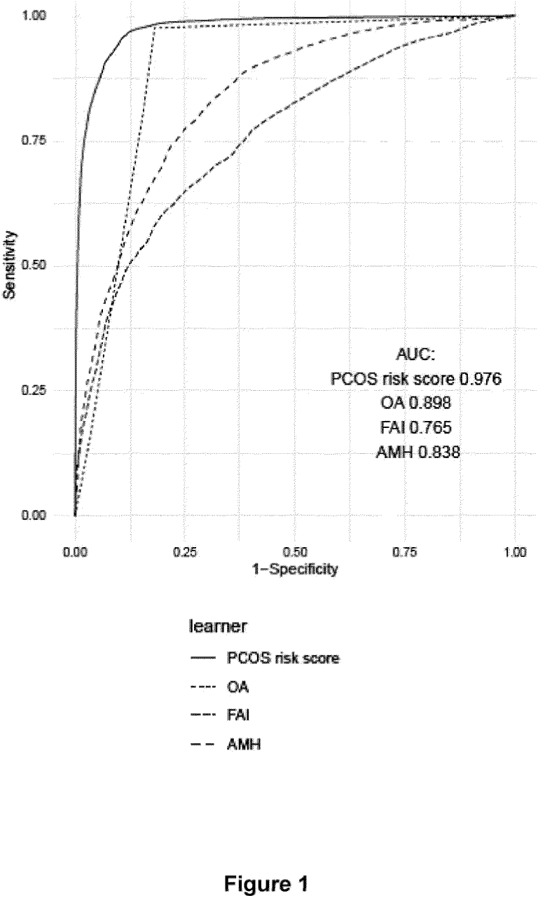

FIG. 1 visualizes the performance of the PCOS risk score based on 1642 controls and 1955 cases. The ROC curves as well as the AUC show superior performance for the PCOS risk score with the variables (age, BMI, OA, FAI, and AMH) as compared to single variables OA, FAI, or AMH alone. The AUC of the PCOS risk score was 0.98 indicating a very good separation between cases and controls, followed by OA (AUC: 0.90). AMH (AUC: 0.84), and FAI (AUC: 0.77). The performance of the three- and four-variable combinations (variables OA, FAI, AMH, and AGE of the PCOS risk or OA, FAI or AMH) was also assessed (see Table 7). The ROC curves as well as the AUC demonstrated that the combined variables of the PCOS risk (age, OA, FAI and AMH) are superior as to using OA, FAI or AMH alone. The AUC of the PCOS risk reaches 98%, indicating that a very good separation between cases and controls can be achieved, followed by OA with 90% and AMH with 84%, The AUC of FAI reaches only about 77%. The three-variable model (combination of OA, AMH and FAI vs OA, FAI or AMH alone) resulted in a PCOS Risk Score AUC of 0.970.

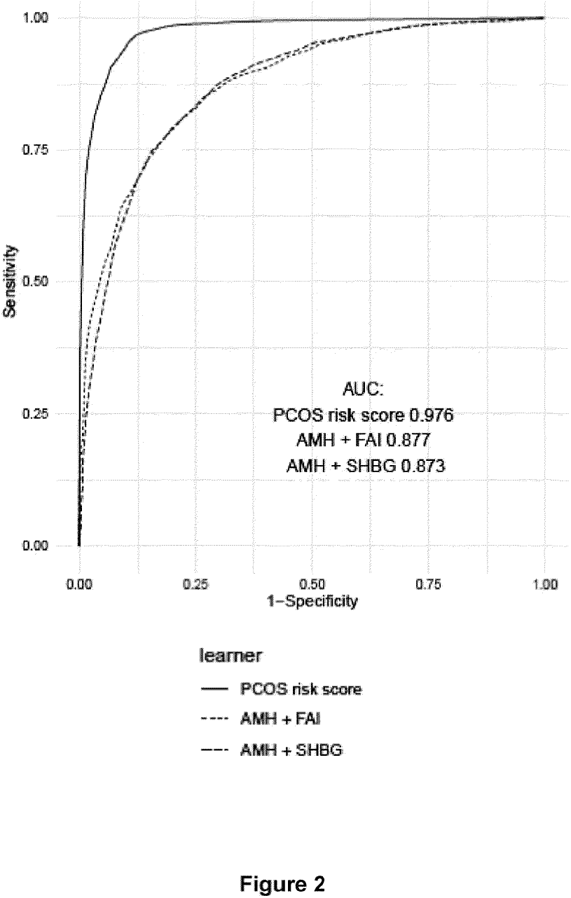

FIG. 2 shows the performance of the PCOS risk score including all variables (age, BMI, OA, FAI, and AMH) compared to a combination of AMH and FAI or a combination of AMH and SHBG (as suggested by Calzada et al.). The ROC curves as well as the AUC show that the variables of the PCOS Risk score (age, BMI, OA, FAI, and AMH) are superior to using AMH combined with either FAI or SHBG. The performance of the model when either all variables of the PCOS risk (except for BMI) or OA, FAI or AMH alone was also assessed (data not shown). The ROC curves as well as the AUC proved that the variables of the PCOS risk (age, OA, FAI and AMH) are superior as to using AMH binarized using a cutoff of 5.03 ng/ml together with either FAI or SHBG. The AUC of the PCOS risk reaches 98%, indicating that a very good separation between PCOS cases and controls without PCOS can be achieved, followed by AMH (binarized using cutoff 5.03 ng/ml) plus SHBG with 82%. The AUC of AMH (binarized using cutoff 5.03 ng/ml) plus FAI reaches only about 76%.

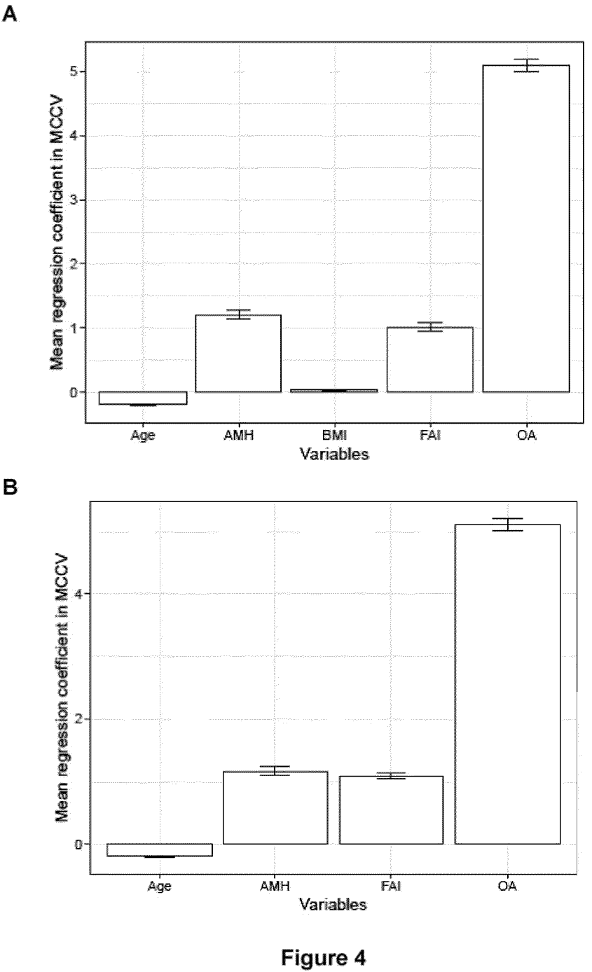

The stability of the weighted logistic regression model was evaluated using 200 MCCV runs and displayed in FIG. 4 as well as Tables 3 and 4. Results indicate that OA has the largest influence on the PCOS risk, followed by AMH and FAI. Small standard deviations indicate quite stable regression coefficients throughout the MCCV runs.

TABLE 3

Table of weights of the PCOS risk derived based on weighted logistic regression using age and BMI as well as OA, FAI and AMH by case-control status based on 1955 cases and 1642 controls.

| Variable | Mean | SD |
|---|---|---|
| FAI | 1.02 | 0.06 |
| AMH | 1.21 | 0.07 |
| Age | -0.19 | 0.01 |
| BMI | 0.04 | 0.01 |
| OA | 5.09 | 0.10 |

TABLE 4

Table of weights of the PCOS risk derived based on weighted logistic regression using age as well as OA, FAI and AMH by case-control status based on 1955 cases and 1642 controls.

| Variable | Mean | SD |
|---|---|---|
| FAI | 1.10 | 0.05 |
| AMH | 1.17 | 0.07 |
| Age | -0.19 | 0.01 |
| OA | 5.11 | 0.09 |

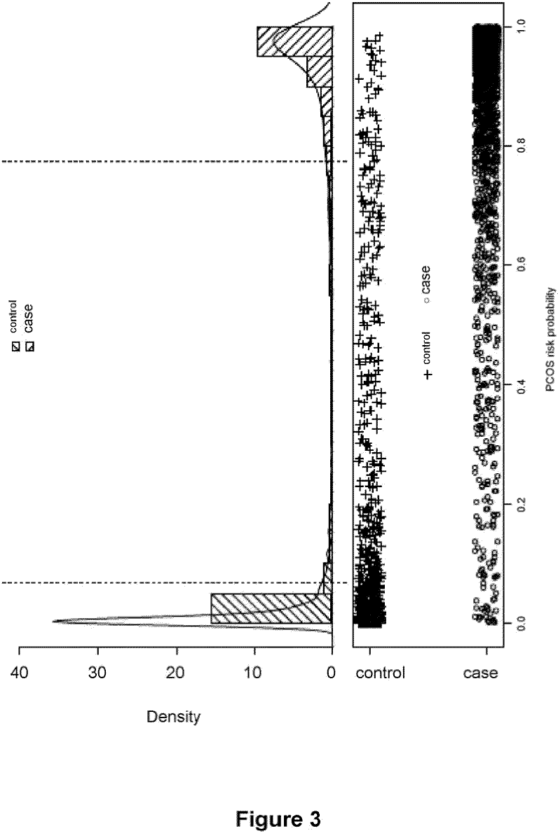

The estimated PCOS risk probabilities resulting from weighted logistic regression using age and BMI as well as OA, FAI and AMH are displayed in FIG. 3 by case-control status. The histogram shows a clear separation of cases and controls with a high estimated risk for the cases and a low risk for the controls. Few subjects are considered as having a moderate risk of PCOS of around 50%. Dashed lines indicate thresholds to group females as low risk, moderate risk and high risk (from left to right) indicating 80% sensitivity and 80% specificity. Similar results were obtained weighted logistic regression using the three-and four-variable models (see Table 7).

### Example 2: Investigation of Risk Thresholds

The PCOS risk derived by means of weighted logistic regression assigns the majority of cases with a high risk of having PCOS, whereas the controls are estimated to have a low risk (see Tables 5 and 6).

TABLE 5

Table of PCOS risk score classification derived based on weighted logistic regression using age as well as OA, FAI and AMH by case-control status based on 1955 cases and 1642 controls.

| Status | PCOS risk low | moderate | high | Total |
|---|---|---|---|---|
| control | 1315 | 276 | 51 | 1642 |
| case | 27 | 365 | 1563 | 1955 |
| Total | 1342 | 641 | 1614 | 3597 |

TABLE 6

Table of PCOS risk score classification derived based on weighted logistic regression using age and BMI as well as OA, FAI and AMH by case-control status based on 1955 cases and 1642 controls.

| Status | PCOS risk low | moderate | high | Total |
|---|---|---|---|---|
| control | 1315 | 279 | 48 | 1642 |
| case | 26 | 366 | 1563 | 1955 |
| Total | 1341 | 645 | 1611 | 3597 |

All in all the PCOS risk without BMI leads to comparable risk classification as the when including BMI into the PCOS risk.

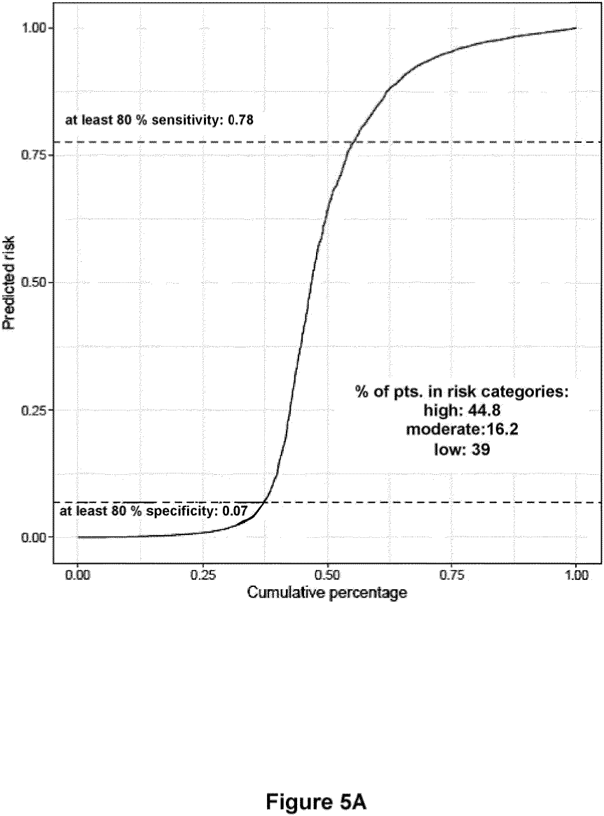

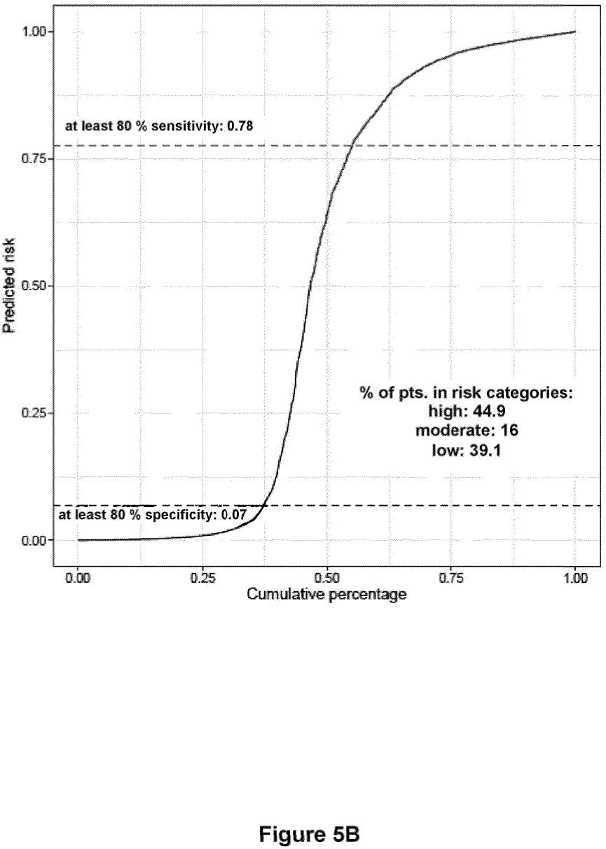

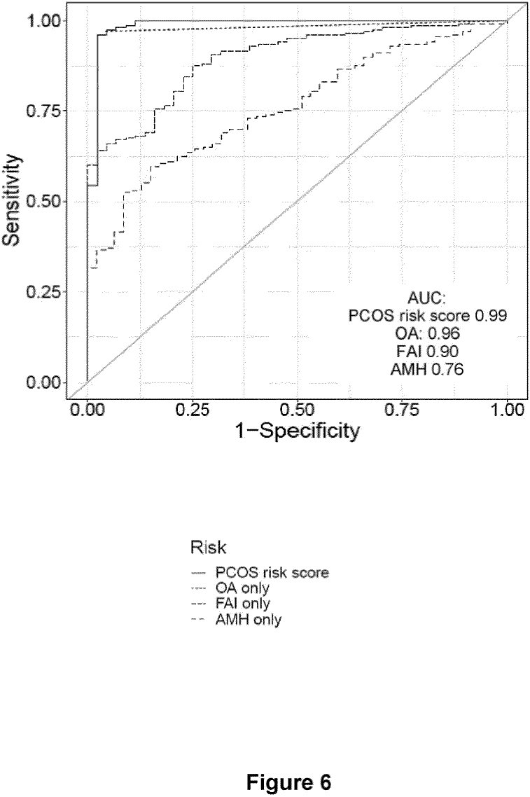

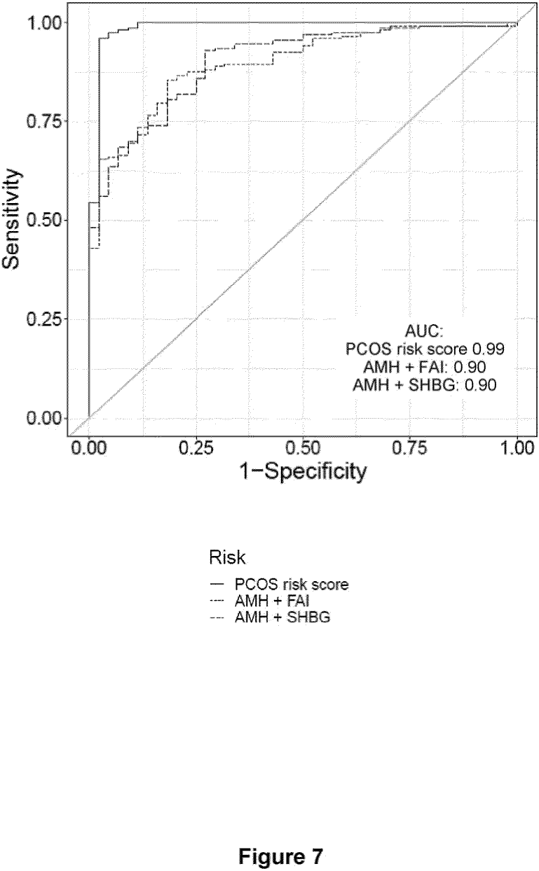

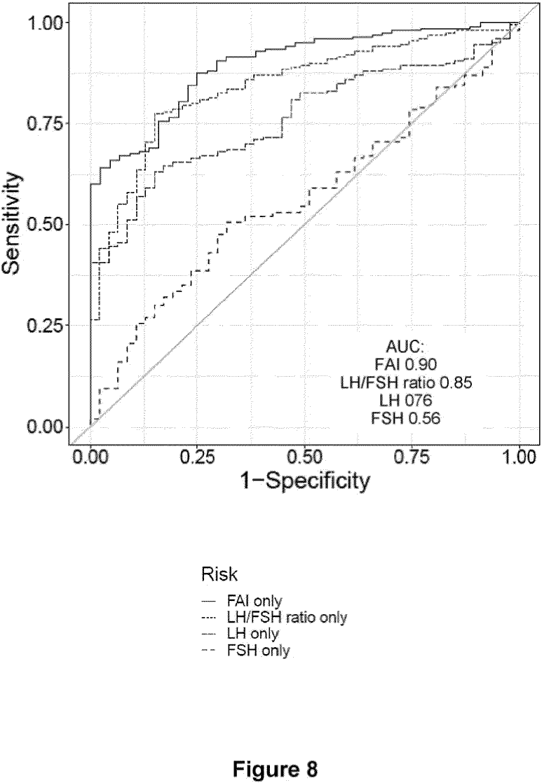

The classification into risk groups based on the predictiveness curve results in risk thresholds of 7% for low risk (specificity ≥80%) and of 78% for high risk (sensitivity ≥ 80%). Based on these thresholds, the low risk group contains approximately 39%, the moderate group 16% and the high risk group 45% of the women based on 1955 cases and 1642 controls (FIGS. 5A and B).

TABLE 7

Table of AUC (area under the curve) resulting from weighted logistic regression evaluated using 200 Monte-Carlo cross-validation runs for 1955 cases and 1642 controls based on different combinations of variables. *: applying a cut-off of 5.03 for AMH as suggested by Mahajan & Kaur 2019

| | AUC |
|---|---|
| PCOS risk score (age, BMI, OA, FAI, and AMH) | 0.98 |
| PCOS risk score (age, OA, FAI, and AMH) | 0.98 |
| PCOS risk score (OA, FAI, and AMH) | 0.97 |
| OA | 0.90 |
| FAI | 0.76 |
| AMH | 0.84 |
| AMH + FAI | 0.88 |
| AMH + SHBG | 0.87 |
| AMH* | 0.72 |
| AMH* + FAI | 0.76 |
| AMH* + SHBG | 0.82 |

### Example 3: Evaluation of the PCOS Risk Score

The performance of the PCOS risk score was evaluated on a second independent sample set of 200 cases and 44 controls.

### Controls

The controls consisted of 44 healthy women between aged 18 - 38 years without having PCOS. The median age was 25.5 years (standard deviation=5.02) and the majority had a normal body mass index (BMI, median=21.9 kg/m², standard deviation=1.88). All women included in this control group had regular cycles based on information of menstrual cycles and/or cycle length. Serum Anti-Mullerian Hormone (AMH) was measured using Elecsys AMH Plus immunoassay. Hyperandrogenism (HA) was derived as the free androgen index (FAI) based on levels of serum testosterone (nmol/L) and serum sex hormone-binding globulin (SHBG) (nmol/L).

$$\text{FAI} \text{=} \text{testosterone} \text{/} \text{SHBG * 100}$$

Testosterone and SHBG levels to depict the FAI were determined by Elecsys Testosterone II (nmol/L) and Elecsys SHBG immunoassays (nmol/L) .

The three serum assays were measured from a serum sample taken at days 1-3 of the menstrual cycle.

### Cases

200 women diagnosed with PCOS aged 20 to 41 years and not using contraceptives were considered. Oligomenorrhea and/or anovulation (OA) was based on information of irregular menstrual cycles and/ or cycle length. Hyperandrogenism (HA) was derived as the free androgen index (FAI) based on levels of serum testosterone (nmol/L) and serum sex hormone-binding globulin (SHBG) (nmol/L):

$$\text{FAI} \text{=} \text{testosterone} \text{/} \text{SHBG * 100}$$

Patients were evaluated for PCOM by an ovarian volume ≥10 mL and/or an antral follicle count (AFC) above threshold based on transvaginal ultrasound examination. Serum Anti-Mullerian hormone (AMH) was measured using the Elecsys AMH Plus immunoassay.

The PCOS cases covered PCOS patients representing the four phenotypes according to the Rotterdam criteria (PCOS Consensus Workshop Group, Fertil Steril 2004;81:19-25).

The following table lists the baseline characteristics for the cases and controls.

TABLE 8

Descriptive statistics of the variables of the PCOS risk score overall and for patients (cases) and controls of the independent second sample set

| | | All (N=244) | Controls (N=44) | Cases (N=200) |
|---|---|---|---|---|
| PCOS Phenotype | A (OA+, HA+, PCOM+) | N=121 (49.6%) | N=0 | N=121 (60.5%) |
| | B (OA+, HA+, PCOM-) | N=6 (2.5%) | N=0 | N=6 (3.0%) |
| | C (OA-, HA+, PCOM+) | N=5 (2.0%) | N=0 | N=5 (2.5%) |
| | D (OA+, HA-, PCOM+) | N=67 (27.5%) | N=0 | N=67 (33.5%) |
| | None | N=1 (0.41%) | N=0 | N=1 (0.50%) |
| OA* | Regular Cycle | N=48 (19.7%) | N=42 (85.5%) | N=6 (3.0%) |
| | OA | N=196 (80.3%) | N=2 (4.5%) | N=194 (97.0%) |
| FAI* | Mean | 5.16 | 1.30 | 6.01 |
| | SD | 5.34 | 0.931 | 5.54 |
| | Median | 3.52 | 0.966 | 4.64 |
| | Q1 ... Q3 | 1.67 ... 6.51 | 0.594 ... 1.65 | 2.40 ... 7.87 |
| | Min ... Max | 0.128 ... 39.7 | 0.129 ... 3.58 | 0.318 ... 39.7 |
| AMH (ng/mL) | Mean | 5.88 | 3.37 | 6.41 |
| | SD | 3.71 | 1.74 | 3.81 |
| | Median | 5.13 | 3.21 | 5.70 |
| | Q1 ... Q3 | 3.16... 7.56 | 2.02 ... 4.47 | 3.54 ... 8.41 |
| | Min ... Max | 0.0455 ... 23.0 | 0.308 ... 7.48 | 0.0455 ... 23.0 |
| Age (years) | Mean | 27.8 | 26.5 | 28.0 |
| | SD | 4.84 | 5.02 | 4.78 |
| | Median | 27.0 | 25.5 | 28.0 |
| | Q1 .. Q3 | 24.0 ... 31 0 | 23.0 ... 30.5 | 24.0...31.0 |
| | Min ... Max | 18.0 ... 41.0 | 18.0 ... 36.0 | 20.0...41.0 |
| BMI | Mean | 25.7 | 22.2 | 26.5 i |
| | SD | 5.71 | 1.88 | 5.97 i |
| | Median | 24.2 | 21.9 | 25.5 |
| | Q1 ... Q3 | 21.5 ... 29.0 | 21.1 ... 23.6 | 22.2... 30.4 |
| | Min ... Max | 16.7 ... 45.3 | 18.2 ... 26.0 | 16.7 ... 45.3 |
| Testosterone (nmol/L)* | Mean | 1.78 | 0.914 | 1.97 |
| | SD | 0.931 | 0.393 | 0.907 |
| | Median | 1.56 | 0.913 | 1.74 |
| | Q1 ...Q3 | 1.09 ... 2.37 | 0.598 ... 1.15 | 1.32 ... 2.58 |
| | Min ... Max | 0.0870 ... 4.79 | 0.0870 ... 1.79 | 0.350 ... 4.79 |
| SHBG (nmol/L)* | Mean | 52.3 | 68.6 | 44.3 |
| | SD | 32.3 | 39.1 | 24.3 |
| | Median | 45.7 | 60.8 | 38.2 |
| | Q1 ...Q3 | 29.4 ... 66.6 | 56.6 ... 111 | 27.0 ... 58.9 |
| | Min ... Max | 8.30 ... 192 | 36.0 ... 192 | 8.30... 152 |

OA: Oligomenorrhea and/or anovulation
FAI: Free Androgen Index; FAI = Testosterone / SHBG *100
BMI. Body Mass Index; BMI = weight (kg) / size (m)²
AMH: Anti-Mullerian Hormone
SHBG: Sex Hormone-Binding Globulin
HA: HyperAndrogenism
PCOM: PolyCystic Ovary Morphology

### Sensitivity and Specificity of the PCOS Risk Score

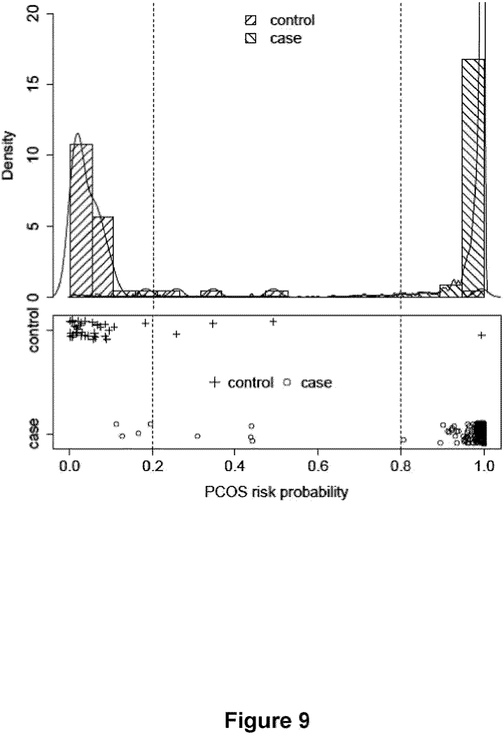

Different thresholds were applied to the independent second sample set of 200 cases and 44 controls. Best results were achieved at fixed risk probability thresholds of 0.2 and 0.8 resulting in 96.0% sensitivity and 90.9% specificity (see FIG. 9).

Moreover, the following table lists the ROC area under the curve values (AUC) for different variable combinations applied to the independent second data set showing a superior performance of the PCOS risk score combination:

TABLE 9

Table of AUC (area under the curve) resulting from weighted logistic regression evaluated using 200 MCCV runs and 100 repetitions for 200 cases and 44 controls using different variable combinations. *: applying a cut-off of 5.03 for AMH as suggested by Mahajan & Kaur 2019

| Variable combination | AUC |
|---|---|
| PCOS risk score (age, BMI, OA, FAI, AMH) | 0.99 |
| OA only | 0.96 |
| FAI only | 0.90 |
| LH/FSH ratio only | 0.85 |
| LH only | 0.76 |
| FSH only | 0.56 |
| AMH only | 0.76 |
| AFC only | 0.82 |
| AMH + FAI | 0.90 |
| AMH + SHBG | 0.90 |
| AMH* | 0.72 |
| AMH*+ FAI | 0.92 |
| AMH* + SHBG | 0.92 |
| PCOS risk score w/o BMI | 0.97 |

### REFERENCES

Calzada et al. “AMH in combination with SHBG for the diagnosis of polycystic ovary syndrome”; J Obstet Gynaecol. 2019, 17:1-7.

Cho et al. “The biological variation of the LH/FSH ratio in normal women and those with Polycystic Ovarian Syndrome”; 2005 Endocrine Abstracts 2005; 9 P80.

Escobar-Morreale H. F., “Polycystic ovary syndrome: definition, aetiology, diagnosis and treatment”; Nature Reviews Endocrinology 2018; Vol 14, 270-284.

Indian et al. “Simplified 4-item criteria for polycystic ovary syndrome: A bridge too far?”; Clin. Endocrinol. (Oxf). 2018; doi: 10.1111/cen.13755.

International evidence-based guideline for the assessment and management of polycystic ovary syndrome 2018.

Mahajan, Nalini, & Kaur, Jasneet. 2019, Establishing an Anti-Muelledan hormone cut-off for diagnosis of polycystic, ovarian syndrome in women of reproductive age-bearing Indian ethnicity using the automated Anti-Muellerian hormone assay. Journal of Human Reproductive Sciences, 12(2), 104-113.

Malini and George “Evaluation of different ranges of LH:FSH ratios in polycystic ovarian syndrome (PCOS) - Clinical based case control study’ General and Comparative Endocrinology 2018; 260: 51-57

Mireya Calzada, Natividad Lopez, Jose A. Noguera, Jaime Mendiola, Ana I, Hernàndez, Shiana Corbalan, Maria Sanchez a Alberto M. Torres (2019): AMH in combination with SHBG for the diagnosis of polycystic ovary syndrome, Journal of Obstetrics and Gynaecology, JOURNAL OF OBSTETRICS AND GYNAECOLOGY

Nicholas et al. ,The utility of Anti-Müllerian Hormone in diagnosing Polycystic Ovary Syndrome amongst women presenting to an infertility clinic”; Hum Reprod. Abstract ESHRE 2014.

Nordenstrom A. and Falhammar H., ,,MANAGEMENT OF ENDOCRINE DISEASE: Diagnosis and management of the patient with non-classic CAH due to 21-hydroxylase deficiency.”; Eur J Endocrinol. 2018; pil: EJE-18-0712.R2. doi:10.1530/EJE-18-0712.

Pepe et al., “integrating the Predictiveness of a Marker with Its Performance as a Classifier”; Am J Epidemiol. 2008, 167(3): 362-368.

Pigny et al. ,,Comparative assessment of five serum antimüllerian hormone assays for the diagnosis of polycystic ovary syndrome.”: Fertil Steril. 2016; 105(4):1063-1069.e3.

R Core Team. 2015. R: A Language and Environment for Statistical Computing. R Foundation for Statistical Computing, Vienna, Austria (https://www.r-project.org/)

Sahmay et al. “Diagnosis of Polycystic Ovary Syndrome: AMH in combination with clinical symptoms”; J Assist Reprod Genet. 2014; 31(2): 213-220.

Scouten, W. H ., “A survey of enzyme coupling techniques.”; Methods in Enzymology 135:30-65. 1987.

Tijssen, P., Practice and theory of enzyme immunoassays, Elsevier Science Publishers B.V., Amsterdam (1990), the whole book, especially pages 43-78 and pages 108-115

Vermeulen, A., L. Verdonck, and J. Kaufman, A critical evaluation of simple methods for the estimation of free testosterone in serum. Journal of Clinical Endocrinology & Metabolism, 1999. 84(10): p. 3666-3672.

Xu. Qing-Song, & Liang, Yi-Zeng. 2001. Monte Carlo cross validation. Chemometrics and Intelligent Laboratory Systems, 56(1), 1-11.

The Rotterdam ESHRE/ASRM-Sponsored PCOS Consensus Workshop Group. Revised 2003 consensus on diagnostic criteria and long-term health risks related to polycystic ovary syndrome Fertil Steril 2004; 61:19-25.

1. A method of assessing a female’s risk of having polycystic ovary syndrome (PCOS), the method comprising
a) providing a data set including
an OA-value reflecting the length of the female’s menstrual cycle and/or the number of the female’s menstrual cycles per year, wherein an increased OA-value relative to the OA-value of a healthy reference population indicates an abnormal menstrual cycle length and/or number,
a HA-value reflecting the female’s androgen status, wherein an increased HA-value relative to the HA-value of a healthy reference population indicates an increased androgen level in the female, and
an AMH-value corresponding to the amount or concentration of anti-Mullerian hormone (AMH) in a sample obtained from the female;
b) processing the data set provided in step a) with a processing unit, wherein the processing comprises combining values of the data set provided in step a) into one combined value;
c) comparing the combined value obtained in step b) to the corresponding combined value as established in a reference population, wherein an increased combined value of the female relative to the combined value of a healthy reference population is indicative of an increased risk of PCOS; and
d) indicating the female’s risk of having PCOS via an indication unit.

2. The method of claim 1, wherein the data set of step a) further includes
a WEIGHT-value reflecting the female’s body weight, wherein an increased WEIGHT-value relative to the WEIGHT-value of a normal weight population indicates an increased body weight; and/or
an AGE-value reflecting the female’s age.

3. The method of claim 1, wherein the HA-value corresponds to
the amount or concentration of free testosterone (FT) in a sample obtained from the female, or
the ratio of the amount or concentration of total testosterone (TT) and the amount or concentration of sex hormone-binding globulin (SHBG) in a sample obtained from the female (TT/SHBG), optionally multiplied by a constant a (a * TT/SHBG).

4. The method of claim 1, wherein in step b) the combined value is a weighted combined value obtained by weighted calculation of the values provided in step a) and in step c) the weighted combined value is compared to the corresponding weighted combined value of a reference population, wherein an increased weighted combined value of the female is indicative of an increased risk of having PCOS.

5. The method of claim 1, wherein
the female has a high risk, if the combined value is above a threshold_{high}; and
the female has a moderate risk, if the combined value is above a threshold_{moderate} and below threshold_{high}; and
the female has a low risk, if the combined value is below threshold_{moderate}.

6. The method of claim 1, wherein one or more values of the reference population and/or the combined value of the reference population are retrieved from a database.

7. The method of claim 1, wherein the data set of step a) further includes a PHE-value reflecting one or more phenotypical characteristics known to be indicative of PCOS, wherein an increased PHE-value relative to the PHE-value of a healthy reference population indicates the presence of one or more phenotypical characteristics known to be indicative of PCOS is reflected by an increased PHE-value, and wherein the phenotypical characteristic is polycystic ovarian morphology (PCOM) and/or hyperandrogenemia hirsutism.

8. The method of claim 1, wherein the sample is a blood sample selected from the group consisting of serum, plasma, and whole blood.

9. The method of claim 1, wherein the female is a human.

10. The method of claim 1, wherein the method further includes determining one or more values of the data set provided in step a).

11. The method of claim 1, wherein the amount or concentration of one or more of the hormone(s) in the female’s sample is measured by an immunoassay and/or mass spectrometry.

12. A kit for assessing a female’s risk of having PCOS, the kit comprising reagents required to specifically measure in a sample obtained from the female
(i) the amount or concentration of FT or (ii) the amount or concentration of TT and the amount or concentration SHBG;
the amount or concentration of AMH; and
optionally the amount or concentration of one or more further hormones indicative of PCOS.

13. (canceled)

14. A computer system comprising:
a) a data set unit comprising computer instructions for providing a data set including
an OA-value reflecting the length of the female’s menstrual cycle and/or the number of the female’s menstrual cycles per year, wherein an increased OA-value relative to the OA-value of a healthy reference population indicates an abnormal menstrual cycle length and/or number;
a HA-value reflecting the female’s androgen status, wherein an increased HA-value relative to the HA-value of a healthy reference population indicates an increased androgen level in the female; and
an AMH-value corresponding to the amount or concentration of AMH in a sample obtained from the female;
b) a processing unit comprising computer instructions for processing the data sets of step a), wherein the processing comprises combining values of the data set provided in step a) into one combined value;
c) a reference data unit comprising computer instructions for
(i) storing and/or retrieving a reference data set including one or more of the reference values as established in a reference population and processing the reference data set into a combined value of the reference population; or
(ii) storing and/or retrieving a combined value of the reference population;
(d) comparing the combined value obtained in step b) to the corresponding combined value of step c), wherein an increased combined value of the female relative to the combined value of a healthy reference population is indicative of an increased risk of PCOS; and
e) an indication unit indicating the female’s risk of having PCOS.

15. (canceled)

16. (canceled)

17. Computer-readable storage medium comprising instructions which, when executed by a computer, cause the computer to carry out steps a), b), c) and d) of the method of claim 1.
